SEQUENCE LISTING (2 sequences)

SEQ ID NO: 1 - protein, 841 aa, Felis catus
  REGION 1..841: note = Description of sequence: T1R1 protein component
MSLPAAHLVGLQLSLSCCWALSCHSTETSADFSLPGDYLLAGLFPLHSDCPGVRHRPTVTLCDRPDSFNGHGYHLFQAMRFGIEEINNSTALLPNVTLGYQLYDVCSESANVYATLNVLSLLGTHHVEIRADPSHYSPAALAVIGPDTTNHAATTAALLSPFLVPLISYEASSVTLGVKRHYPSFLRTIPSDKHQVEAMVLLLQSFGWVWISVVGSDGDYGQLGVQALEEQATQQGICVAFKDIIPFSARPGDERMQGIMHHLARARTTVVVVFSSRQLARVFFESVVLANLTAKVWIASEDWAISRHISNVPGIQGIGTVLGVAIQQRLVPGLKEFEEAYVQADKGAPGPCSRTSECSSNQLCRECRAFTAEQMPTLGAFSMSSAYNAYRAVYAVAHGLHQLLGCASGACSRDRVYPWQLLEQIRKVNFLLHKDTVRFNDNGDPLSGYDIIAWDWSGPKWNFRVIGSSMWPPVQLDINKTKIRWHGKDNQVPKSVCSSDCLEGHQRVISGFYHCCFECVPCEAGSFLNKSDLHSCQPCGKEEWAPAGSETCFPRTVVFLTWHETISWVLLAANTLLLLLVTGTAGLFAWHLDTPVVKSAGGRLCFFMLGSLAGGSCGLYGFFGEPTLPTCLLRQSLLALGFAIFLSCLTIRSFQLVFIFKFSAKVPTFYRAWVQNHGPGLFVVISSMAQLLICLTWLAVWTPLPTREYQRFPQLVVLDCTEANSPGFMLAFAYNGLLSVSAFACSYLGKDLPENYNEAKCVTFSLLLNFVSWIAFFTTASVYQGKYLPAVNVLAALSSLSGGFSGYFLPKCYVILCRPDLNSTEHFQASIQEYTRRCGST

SEQ ID NO: 2 - protein, 865 aa, Felis catus
  REGION 1..865: note = Description of sequence: T1R3 protein component
MPGLALLGLTALLGLTALLDHGEGATSCLSQQLRMQGDYVLGGLFPLGSAEGTGLGDGLQPNATVCTRFSSLGLLWALAVKMAVEEINNGSALLPGLHLGYDLFDTCSEPMVAMKPSLVFMAKAGSCSIAAYCNYTQYQPRVLAVIGPHSSELALVTGKFFSFFLVPQVSYGASTDRLSNREIFPSFFRTVPSDQVQVAAMVELLQELGWNWVAAVGSDDEYGRQGLSLFSGLASARGICIAHEGLVPLPPGSLRLGALQGLLRQVNQSSVQVVVLFSSAHAARTLFSYSIRCKLSPKVWVASEAWLTSDLVMTLPGMPGVGTVLGFLQQGAPMPEFPSYVRTRLALAADPAFCASLDAEQPGLEEHVVGPRCPQCDHVTLENLSAGLLHHQTFAAYAAVYGVAQALHNTLRCNASGCPRREPVRPWQLLENMYNVSFRARGLALQFDASGNVNVDYDLKLWVWQDPTPELRTVGTFKGRLELWRSQMCWHTPGKQQPVSQCSRQCKEGQVRRVKGFHSCCYDCVDCKAGSYQRNPDDLLCTQCDQDQWSPDRSTRCFARKPMFLAWGEPAVLLLLALLALALGLALAALGLFLWHSDSPLVQASGGPRACFGLACLGLVCLSVLLFPGQPGPASCLAQQPLFHLPLTGCLSTLFLQAAEIFVGSELPPSWAEKMRGRLRGPWAWLVVLLAMLAEAALCAWYLVAFPPEVVTDWRVLPTEALVHCHVHSWISFGLVHATNAMLAFLCFLGTFLVQSRPGRYNGARGLTFAMLAYFITWISFVPLFANVHVAYQPAVQMGTILLCALGILATFHLPKCYLLLQRPELNTPEFFLEDNARAQGSSWGQGRGESGQKQVTPDPVTSPQ

(19) United States
(12) Patent Application Publication
(10) Pub. No.: US 2026/0041730 A1
(43) Pub. Date: Feb. 12, 2026

(54) COMPOSITIONS FOR DRINKING WATER

(71) Applicant: MARS, INCORPORATED, McLean, VA (US)

(72) Inventors: Scott MCGRANE, Melton Mowbray (GB); Carlos HERNANGOMEZ DE ALVARO, Melton Mowbray (GB); Anne Marie BAKKE, Melton Mowbray (GB); Sofia SCHAUF BAILON, Melton Mowbray (GB); Anni LAFFITTE, Melton Mowbray (GB); Annabelle GOYON, Aimargues (FR); Yann QUEAU, Aimargues (FR); Laurence LE VERGER, Aimargues (FR); Dominique LOBBE, Aimargues (FR); Mathieu MAO, Aimargues (FR)

(73) Assignee: MARS, INCORPORATED, McLean, VA (US)

(21) Appl. No.: 18/849,847

(22) PCT Filed: Mar. 24, 2023
(86) PCT No.: PCT/US2023/016247

§ 371 Date: Sep. 23, 2024

Related U.S. Application Data
(60) Provisional application No. 63/323,309, filed on Mar. 24, 2022.

(51) Int. Cl. A61K 38/17; A23K 10/16; A23K 20/121; A23K 20/147; A23K 20/153; A23K 20/26; A23K 50/40; A61K 31/34; A61K 31/401; A61K 31/4172; A61K 31/708; A61K 33/42; A61K 36/064; A61P 13/02

(52) U.S. Cl. CPC A61K 38/1709; A23K 10/16; A23K 20/121; A23K 20/147; A23K 20/153; A23K 20/26; A23K 50/40; A61K 31/34; A61K 31/401; A61K 31/4172; A61K 31/708; A61K 33/42; A61K 36/064; A61P 13/02

(57) ABSTRACT

The present disclosure relates to flavor compositions and pet food products for preventing and/or treating chronic kidney disease (CKD) and/or urinary tract disease in an animal. In certain embodiments, the flavor compositions and pet food products have enhanced palatability.

### CROSS-REFERENCES TO RELATED APPLICATIONS

This application is a U.S. National Stage Patent Application under 35 U.S.C. § 371 of International Patent Application No. PCT/US2023/016247, filed on Mar. 24, 2023, which claims the benefit of priority to U.S. Provisional Patent Application Ser. No. 63/323,309, filed on Mar. 24, 2022, the contents of which are incorporated herein by reference in their entirety entireties, and to each of which priority is claimed.

### SEQUENCE LISTING

This application contains a Sequence Listing which has been submitted electronically in XML format and is hereby incorporated by reference in its entirety. Said XML copy, created on Mar. 20, 2023, is named 069269.0609_ST26.xml, and is 5,147 bytes in size.

### FIELD

The presently disclosed subject matter relates to compositions and/or compounds that promote water intake in cats. The increased water intake has benefits for maintenance and improvement of urinary tract and kidney function and their health.

### BACKGROUND

Cats (Felis catus) are obligate carnivores (Bradshaw et al., Comparative Biochemistry and Physiology Part A: Physiology 114, no. 3 (1996): 205-209) and were originally desert animals; as such, they tend to satisfy most of their water requirements by eating prey, which typically contains in excess of 75% moisture, and they will voluntarily drink only small amounts of water by nature (Burger et al., Nutrition of the dog and cat, pp 145-156. (1980). Ed. R S Anderson. Oxford. Pergamon Press.). From a physiological point of view, cats are able to produce much more concentrated urine (specific gravity [SG] of up to 1.095 g/mL) than either humans (up to 1.040 g/mL) or dogs (up to 1.065 g/mL) (Markwell et al., Proceedings from the 9th International Symposium on Urolithiasis. 2000; Osborne and Nwaokorie. When urine specific gravity values go awry in veterinary practice. DVM360 Magazine, Jul. 1, 2013), thus allowing them to cope with water restriction by reducing water losses through urine. However, because this may lead to an increased risk of kidney and urinary tract pathologies, many dietary strategies in cats are targeted to increase water intake in order to prevent or reduce urine mineral saturation and crystallization, which is a similar approach as that taken in humans (Borghi et al., The Journal of urology 155.3 (1996): 839-843; Borghi et al., Nephron 81. Suppl. 1 (1999): 31-37). Thus, there is a need for the development of novel preventive and therapeutic strategies against urinary tract pathologies, which can also help prevent secondary kidney health challenges such as chronic kidney disease (Bartlett et al., Veterinary Medicine International 2010 (2010): 957570).

### SUMMARY OF THE INVENTION

The present disclosure relates to compositions and/or compounds that promote water intake in cats.

In one aspect, the present disclosure provides a method of preventing and/or treating chronic kidney disease in an animal in need thereof. In another aspect, the present disclosure provides a method of preventing and/or treating urinary tract disease in an animal in need thereof. In certain embodiments, the methods comprise administering a pet food product comprising a first amino acid capable of activating an umami receptor and a second amino acid not capable of activating the umami receptor.

In a further aspect, the present disclosure provides a method of increasing water consumption by an animal. Also, in one aspect, the present disclosure provides a method of increasing hydration of an animal. Additionally, in one aspect, the present disclosure provides a method of preventing and/or treating chronic kidney disease. Moreover, in another aspect, the present disclosure provides a method of preventing and/or treating urinary tract disease in an animal. In certain embodiments, the methods comprise administering a pet food product comprising a milk protein and a nucleotide or derivative thereof.

In certain embodiments, the first amino acid is alanine, histidine, glycine, serine, tyrosine, or a mixture thereof. In certain embodiments, the second amino acid is proline, hydroxyproline, valine, threonine, lysine, or a mixture thereof. In certain embodiments, the first amino acid is alanine, histidine, glycine, serine, tyrosine, or a mixture thereof; and the second amino acid is proline, hydroxyproline, valine, threonine, lysine, or a mixture thereof. In certain embodiments, the first amino acid is histidine and any of the listed second amino acids. In certain embodiment, the first amino acid is histidine and the second amino acid is proline, hydroxyproline, valine, threonine, lysine, or a mixture thereof. In certain embodiments, the first amino acid is alanine, histidine, glycine, serine, tyrosine, or a mixture thereof, and the second amino acid is proline. In certain embodiments, the first amino acid and the second amino acid are at a molar ratio of about 1:1. In certain embodiments, the first amino acid and the second amino acid are at a molar ratio of about 1:1.3. In certain embodiments, each of the first and second amino acids is in an amount of from about 1 mM to about 1 M. In certain embodiments, each of the first and second amino acids is in an amount of about 100 mM. In certain embodiments, each of the first and second amino acids is in an amount of from about 20 mM to about 50 mM.

In certain embodiments, the pet food product does not comprise a phosphate or a derivative thereof and/or does not comprise a furan or a derivate thereof. In certain embodiments, the pet food product further comprises a yeast extract.

In certain embodiments, the milk protein is a casein or a derivative thereof. In certain embodiments, the milk protein is at an amount of from about 1% to about 10% by weight. In certain embodiments, the milk protein is at an amount of about 3% by weight.

In certain embodiments, the nucleotide or derivative thereof is AMP, UMP, GMP, IMP, CMP, or a mixture thereof. In certain embodiments, the mixture of nucleotides comprises GMP and IMP. In certain embodiments, the nucleotide or derivative thereof is GMP. In certain embodiments, the nucleotide or derivative thereof is IMP.

In certain embodiments, the nucleotide or derivative thereof is obtained from a yeast extract. In certain embodiments, the yeast extract is a Kluyveromyces yeast extract, a Torula yeast extract, or a Saccharomyces cerevisae yeast extract.

In certain embodiments, the nucleotide or derivative thereof is at an amount of from about 1 mM to about 100 mM. In certain embodiments, the nucleotide or derivative thereof is at an amount of about 5 mM.

In certain embodiments, the pet food product further comprises a furan or derivative thereof. In certain embodiments, the furan or derivative thereof is a furaneol. In certain embodiments, the furan or derivative thereof is at an amount of from about 1 ppm to about 100 ppm. In certain embodiments, the furan or derivative thereof is at an amount of about 4 ppm.

In certain embodiments, the pet food product further comprises a phosphate or derivative thereof. In certain embodiments, the phosphate is a pyrophosphate. In certain embodiments, the phosphate is at an amount of from about 1 mM to about 1 M. In certain embodiments, the phosphate is at an amount of about 10 mM.

In certain embodiments, the pet food product further comprises a furan or derivative thereof and a phosphate or derivative thereof. In certain embodiments, the pet food product further comprises one or more vitamins, minerals, antioxidants, thickening agents, or a combination thereof. In certain embodiments, the pet food product is a drinking water. In certain embodiments, the drinking water is a packaged drinking water. In certain embodiments, the animal is a feline.

In one aspect, the present disclosure provides a pet food product for use in the prevention and/or treatment of chronic kidney disease in an animal in need thereof, wherein the pet food product comprises a first amino acid capable of activating an umami receptor and a second amino acid not capable of activating the umami receptor.

In another aspect, the present disclosure provides a pet food product for use in the prevention and/or treatment of urinary tract disease in an animal in need thereof, wherein the pet food product comprises a first amino acid capable of activating an umami receptor and a second amino acid not capable of activating the umami receptor.

In a further aspect, the present disclosure provides a pet food product for use in increasing water consumption by an animal, wherein the pet food product comprises a milk protein and a nucleotide or derivative thereof.

Additionally, in one aspect, the present disclosure provides a pet food product for use in increasing hydration of an animal, wherein the pet food product comprises a milk protein and a nucleotide or derivative thereof.

Moreover, in one aspect, the present disclosure provides a pet food product for use in preventing and/or treating chronic kidney disease in an animal in need thereof, wherein the pet food product comprises a milk protein and a nucleotide or derivative thereof.

In another aspect, the present disclosure provides a pet food product for use in preventing and/or treating urinary tract disease in an animal in need thereof, wherein the pet food product comprises a milk protein and a nucleotide or derivative thereof.

In certain embodiments, the first amino acid is alanine, histidine, glycine, serine, tyrosine, or a mixture thereof. In certain embodiments, the second amino acid is proline, hydroxyproline, valine, threonine, lysine, or a mixture thereof. In certain embodiments, the first amino acid is histidine and the second amino acid is proline. In certain embodiments, the first amino acid is tyrosine and the second amino acid is lysine. In certain embodiments, the first amino acid and the second amino acid are at a molar ratio of about 1:1. In certain embodiments, the first amino acid and the second amino acid are at a molar ratio of about 1:1.3. In certain embodiments, each of the first and second amino acids is in an amount of from about 1 mM to about 1 M. In certain embodiments, each of the first and second amino acids is in an amount of about 100 mM. In certain embodiments, each of the first and second amino acids is in an amount of from about 20 mM to about 50 mM.

In certain embodiments, the pet food product does not comprise a phosphate or a derivative thereof and/or does not comprise a furan or a derivative thereof. In certain embodiments, the pet food product further comprises a yeast extract.

In certain embodiments, the milk protein is a casein or a derivative thereof. In certain embodiments, the milk protein is at an amount of from about 1% to about 10% by weight. In certain embodiments, the milk protein is at an amount of about 3% by weight.

In certain embodiments, the nucleotide or derivative thereof is AMP, UMP, GMP, IMP, CMP, or a mixture thereof. In certain embodiments, the nucleotide or derivative thereof is obtained from a yeast extract. In certain embodiments, the yeast extract is a Kluyveromyces yeast extract, a Torula yeast extract, or a Saccharomyces cerevisae yeast extract. In certain embodiments, the nucleotide or derivative thereof is at an amount of from about 1 mM to about 100 mM. In certain embodiments, the nucleotide or derivative thereof is at an amount of about 5 mM.

In certain embodiments, the pet food product further comprises a furan or derivative thereof. In certain embodiments, the furan or derivative thereof is a furaneol. In certain embodiments, the furan or derivative thereof is at an amount of from about 1 ppm to about 100 ppm. In certain embodiments, the furan or derivative thereof is at an amount of about 4 ppm.

In certain embodiments, the pet food product further comprises a phosphate or derivative thereof. In certain embodiments, the phosphate is a pyrophosphate. In certain embodiments, phosphate is at an amount of from about 1 mM to about 1 M. In certain embodiments, the phosphate is at an amount of about 10 mM.

In certain embodiments, the pet food product further comprises one or more vitamins, minerals, antioxidants, thickening agents, or a combination thereof. In certain embodiments, the pet food product is a drinking water. In certain embodiments, the drinking water is a packaged drinking water. In certain embodiments, the animal is a feline.

In another aspect, the present disclosure provides a flavor composition comprising:
a) a first amino acid capable of activating an umami receptor and a second amino acid not capable of activating the umami receptor;
b) a first amino acid capable of activating an umami receptor, a second amino acid not capable of activating the umami receptor, and a yeast extract;
c) a first amino acid capable of activating an umami receptor, a second amino acid not capable of activating the umami receptor, a yeast extract, a mineral, and a phosphate;
d) a first amino acid capable of activating an umami receptor, a second amino acid not capable of activating the umami receptor, a yeast extract, a mineral, a phosphate, and a thickening agent;
e) a milk protein and a nucleotide or derivative thereof;
f) a milk protein, a nucleotide or derivative thereof and a furan or derivative thereof; or
g) a milk protein, a nucleotide or derivative thereof, a furan or derivative thereof and a phosphate or derivative thereof.

In certain embodiments, the first amino acid is histidine and the second amino acid is proline. In certain embodiments, the first amino acid and the second amino acid are at a molar ratio of about 1:1 or about 1:1.3. In certain embodiments, the milk protein is a casein or a derivative thereof. In certain embodiments, the nucleotide or derivative thereof is AMP, UMP, GMP, IMP, CMP, or a mixture thereof. In certain embodiments, the nucleotide or derivative thereof is GMP. In certain embodiments, the nucleotide or derivative thereof is IMP. In certain embodiments, the mixture of nucleotides comprises GMP and IMP. In certain embodiments, the furan or derivative thereof is a furaneol. In certain embodiments, the phosphate is a pyrophosphate.

### BRIEF DESCRIPTION OF THE DRAWINGS

FIG. 1 shows the average difference in intake (g) for flavor composition solutions versus water with 95.0% confidence intervals (Dunnett's test) for Example 1.

FIG. 2 shows weighted average intakes by flavor compositions (95% confidence intervals are based on Tukey's test) for Example 1. The means were all significantly different.

FIG. 3 shows mean free water intake (g) for water baselines 1 and 2 with 95.0% confidence intervals (Diff. 21.9 g, p-value 0.074) for Example 2.

FIG. 4 shows mean daily free water intake from the flavor compositions tested and baseline water with 95.0% confidence intervals using Dunnett's test for Example 2.

FIG. 5 shows mean total water intake from the flavor composition tested and water with 95.0% confidence intervals expressed as g intake per Kg metabolic body weight (g/Kg BW^{0.711}) using Dunnett's test for Example 2.

FIG. 6 shows least square means of diet-only or total caloric intake when cats were offered flavor composition F supplement or not.

FIG. 7 shows least square means of body weights when cats were offered the two (2) diets with or without flavor composition F supplement.

FIG. 8 shows least square means of sodium intake when cats were offered the two (2) diets with or without flavor composition F supplement.

FIG. 9 shows flavor composition F volume intake in ml/kg/d per cats for the two (2) diets.

FIG. 10 shows least square means of flavor composition F supplement volume intake according to the diet.

FIG. 11 shows least square means of ingested volume of water and flavor composition F when cats were offered the same.

FIG. 12A shows urinary volume of cats offered flavor composition F or controls, according to the diet. FIG. 12B shows details of data depicted in FIG. 12A.

FIGS. 13A and 13B show urinary specific gravity when flavor composition F was offered.

FIG. 14A shows urine pH of cats offered flavor composition F or controls, according to the diet. FIG. 14B shows details of data depicted in FIG. 14A.

FIG. 15 shows relative supersaturation (RSS) MAP box plot of cats offered flavor composition F or controls, according to the diet.

FIG. 16A shows least square means of calcium oxalate relative supersaturation (RSS CaOx) of cats offered flavor composition F or controls, according to the diet. FIG. 16B shows details of data depicted in FIG. 16A. FIGS. 16C and 16D show correlation between intake of flavor composition F and RSS CaOx value.

FIG. 17 shows percent change of calcium oxalate relative supersaturation (RSS CaOx) after offering flavor composition F with dry diet intended for urinary tract disease (labeled as “urinary diet”), plotted against the consumption of flavor composition F.

FIG. 18 shows percent change of calcium oxalate relative supersaturation (RSS CaOx) after offering flavor composition F with Regular diet, plotted against the consumption of flavor composition F.

### DETAILED DESCRIPTION

To date, there remains a need for a flavor composition that can provide a desired level of enhanced and targeted palatability of water and/or water-based products. The present disclosure relates to flavor compositions that can be used to increase total water intake in cats. By increasing the water intake in cats, the presently disclosed flavor compositions can be used for prevention and treatment of certain common renal and urinary tract diseases, e.g., chronic kidney disease. For purposes of clarity of disclosure and not by way of limitation, the detailed description is divided into the following subsections:
1. Definitions;
2. Flavor Compositions;
3. Pet Food Products;
4. Formulations and Delivery Systems; and
5. Methods of Treatment.

### 1. Definitions

The terms used in this specification generally have their ordinary meanings in the art, within the context of this invention and in the specific context where each term is used. Certain terms are discussed below, or elsewhere in the specification, to provide additional guidance to the practitioner in describing the methods and compositions of the invention and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.”

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of”, and “consisting essentially of,” the embodiments or elements presented herein, whether explicitly set forth or not.

As used herein, “taste” refers to a sensation caused by activation of receptor cells in a subject's taste buds. In certain embodiments, the taste can be selected from the group consisting of sweet, sour, salt, bitter, kokumi, and umami. In certain embodiments, “taste” can include free fatty acid taste. See, e.g., Cartoni et al., J. of Neuroscience, 30(25): 8376-8382 (2010), the contents of which are incorporated herein by reference. In certain embodiments, a taste is elicited in a subject by a “tastant.” In certain embodiments, a tastant can be a synthetic tastant. In certain embodiments, the tastant is obtained or prepared from a natural source.

As used herein, “taste profile” refers to a combination of tastes, such as, for example, one or more of a sweet, sour, salt, bitter, umami, kokumi and free fatty acid taste. In certain embodiments, a taste profile is produced by one or more tastant that is present in a composition at the same or different concentrations. In certain embodiments, a taste profile refers to the intensity of a taste or combination of tastes, for example, a sweet, sour, salt, bitter, umami, kokumi and free fatty acid taste, as detected by a subject or any assay known in the art. In certain embodiments, modifying, changing, or varying the combination of tastants in a taste profile can change the sensory experience of a subject.

As used herein, “flavor” refers to one or more sensory stimuli, such as, for example, one or more of taste (gustatory), smell (olfactory), touch (tactile) and temperature (thermal) stimuli. In certain non-limiting embodiments, the sensory experience of a subject exposed to a flavor can be classified as a characteristic experience for the particular flavor. For example, a flavor can be identified by the subject as being, but not limited to, a floral, citrus, berry, nutty, caramel, chocolate, peppery, smoky, cheesy, meaty, etc., flavor. As used herein, a flavor composition can be selected from a liquid, solution, dry powder, spray, paste, suspension, and any combination thereof. The flavor can be a natural composition, an artificial composition, a nature identical, or any combination thereof.

As used herein, “flavor profile” refers to a combination of sensory stimuli, for example, tastes, such as sweet, sour, bitter, salty, umami, kokumi and free fatty acid tastes, and/or olfactory, tactile and/or thermal stimuli. In certain embodiments, the flavor profile comprises one or more flavors which contribute to the sensory experience of a subject. In certain embodiments, modifying, changing, or varying the combination of stimuli in a flavor profile can change the sensory experience of a subject.

As used herein “admixing,” for example, “admixing the flavor composition or combinations thereof of the present application with water” or “admixing the compound with water,” refers to the process where the flavor composition, or individual components of the flavor composition, is mixed with or added to the completed product or mixed with some or all of the components of the product during product formation or some combination of these steps. When used in the context of admixing, the term “product” refers to the product or any of its components. This admixing step can include a process selected from the step of adding the flavor composition to the product, spraying the flavor composition on the product, dissolving the flavor composition on the product, suspending the product in the flavor composition, painting the flavor composition on the product, pasting the flavor composition on the product, encapsulating the product with the flavor composition, mixing the flavor composition with the product and any combination thereof. The flavor composition can be a solution, liquid, dry powder, spray, paste, suspension, and any combination thereof.

As used herein, “ppm” means parts-per-million and is a weight relative parameter. A part-per-million is a microgram per gram, such that a component that is present at 10 ppm is present at 10 micrograms of the specific component per 1 gram of the aggregate mixture.

As used herein, “palatability” can refer to the overall willingness of a human or non-human animal, for example, a companion animal, to eat a certain food product. Increasing the “palatability” of a food product can lead to an increase in the enjoyment and acceptance of the food by the human or non-human animal to ensure the human or non-human animal eats a “healthy amount” of the food. Decreasing the “palatability” of a food product can lead to a decrease in the enjoyment and acceptance of the food by the human or non-human animal. The term “healthy amount” of a food as used herein refers to an amount that enables the human or non-human animal to maintain or achieve an intake contributing to its overall general health in terms of micronutrients, macronutrients and calories, for example, such as set out in the “Nutrient requirements of dogs and cats” (NRC, NRC. “Nutrient requirements of dogs and cats.” National Research Council of the National Academies (2006)). In certain embodiments, “palatability” can mean a relative preference of a human or non-human animal for one food product over another. For example, when a human or non-human animal shows a preference for one of two or more food products, the preferred food product is more “palatable,” and has “enhanced palatability.” In certain embodiments, the relative palatability of one food product compared to one or more other food products can be determined, for example, in side-by-side, free-choice comparisons, e.g., by relative consumption of the food products, or other appropriate measures of preference indicative of palatability. Palatability can be determined by a standard testing protocol in which the animal has equal access to both food products such as a test called “two-bowl test” or “versus test.” Such preference can arise from any of the animal's senses, but can be related to, inter alia, taste, aftertaste, smell, mouth feel and/or texture. In certain embodiments, palatability can be determined by a monadic testing protocol as described in Example 1 and Example 2.

The term “pet food” or “pet food product” or “final pet food product” means a product or composition that is intended for consumption by a companion animal, such as cats, dogs, guinea pigs, rabbits, birds and horses. For example, but not by way of limitation, the companion animal can be a “domestic” dog, e.g., Canis lupus familiaris. In certain embodiments, the companion animal can be a “domestic” cat such as Felis domesticus. A “pet food” or “pet food product” includes any food, feed, snack, food supplement, liquid, beverage, treat, toy (chewable and/or consumable toys), meal substitute or meal replacement.

As used herein “nutritionally-complete” refers to pet food product that contains all known required nutrients for the intended recipient of the pet food product, in appropriate amounts and proportions based, for example, on recommendations of recognized or competent authorities in the field of companion animal nutrition. Such foods are therefore capable of serving as a sole source of dietary intake to maintain life, without the addition of supplemental nutritional sources.

As used herein “flavor composition” refers to at least one compound or biologically acceptable salt thereof that modulates, including enhancing, multiplying, potentiating, decreasing, suppressing, or inducing, the tastes, smells, flavors and/or textures of a natural or synthetic tastant, flavoring agent, taste profile, flavor profile and/or texture profile in an animal or a human. In certain embodiments, the flavor composition comprises a combination of compounds or biologically acceptable salts thereof. In certain embodiments, the flavor composition includes one or more excipients.

As used herein, the term “umami receptor” refers to a G protein coupled receptor (GPCR), for example, a T1R1/T1R3 GPCR. The umami receptor can be for example, a cat, dog, human or non-human mammal umami receptor.

As used herein, the terms “modulates” or “modifies” refers to an increase or decrease in the amount, quality or effect of a particular activity of a receptor and/or an increase or decrease in the expression, activity or function of a receptor. “Modulators,” as used herein, refer to any inhibitory or activating compounds identified using in silico, in vitro and/or in vivo assays for, e.g., agonists, antagonists, allosteric modulators and their homologs, including fragments, variants and mimetics.

“Inhibitors” or “antagonists,” as used herein, refer to modulating compounds that reduce, decrease, block, prevent, delay activation, inactivate, desensitize or down regulate the biological activity and/or expression of a receptor or pathway of interest. The term “antagonist” includes full, partial, and neutral antagonists as well as inverse agonists.

“Inducers,” “activators” or “agonists,” as used herein, refer to modulating compounds that increase, induce, stimulate, open, activate, facilitate, enhance activation, sensitize, or upregulate a receptor or pathway of interest. The term “agonist” includes full and partial agonists.

“Allosteric modulators” as used herein, refer to “positive allosteric modulators” and “negative allosteric modulators.” “Positive allosteric modulators” (also known as “PAM”) refer to modulating compounds that increase, induce, stimulate, open, activate, facilitate, enhance activation, sensitize or up regulate a receptor or pathway of interest caused by the binding of a different compound to the receptor. “Negative allosteric modulators” refer to modulating compounds that reduce, decrease, block, prevent, delay activation, inactivate, desensitize or down regulate the biological activity and/or expression of a receptor or pathway of interest caused by the binding of a different compound to the receptor.

The term “nucleic acid molecule” and “nucleotide sequence,” as used herein, refers to a single or double stranded covalently linked sequence of nucleotides in which the 3′ and 5′ ends on each nucleotide are joined by phosphodiester bonds. The nucleic acid molecule can include deoxyribonucleotide bases or ribonucleotide bases and can be manufactured synthetically in vitro or isolated from natural sources.

The terms “polypeptide,” “peptide,” “amino acid sequence” and “protein,” used interchangeably herein, refer to a molecule formed from the linking of at least two amino acids. The link between one amino acid residue and the next is an amide bond and is sometimes referred to as a peptide bond. A polypeptide can be obtained by a suitable method known in the art, including isolation from natural sources, expression in a recombinant expression system, chemical synthesis, or enzymatic synthesis. The terms can apply to amino acid polymers in which one or more amino acid residue is an artificial chemical mimetic of a corresponding naturally occurring amino acid, as well as to naturally occurring amino acid polymers and non-naturally occurring amino acid polymers.

The term “amino acid,” as used herein, can be naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate and O-phosphoserine. Amino acid analogs and derivatives can refer to compounds that have the same basic chemical structure as a naturally occurring amino acid, i.e., a carbon that is bound to a hydrogen, a carboxyl group, an amino group and an R group, e.g., homoserine, norleucine, methionine sulfoxide and methionine methyl sulfonium. Such analogs can have modified R groups (e.g., norleucine) or modified peptide backbones, but retain the same basic chemical structure as a naturally occurring amino acid. Amino acid mimetics means chemical compounds that have a structure that is different from the general chemical structure of an amino acid, but that function in a manner similar to a naturally occurring amino acid. Non-limiting examples of amino acid include tryptophan, phenylalanine, histidine, glycine, cysteine, alanine, tyrosine, serine, methionine, asparagine, leucine, asparagine, threonine, isoleucine, proline, glutamic acid, aspartic acid, hydroxyl proline, arginine, cystine, glutamine, lysine, valine, ornithine, taurine, and combinations thereof.

As used herein, the term “amino acid source” means a material containing amino acids. In certain non-limiting embodiments, the amino acid source can include or be derived from plant proteins, animal proteins, proteins from single cell organisms, and free amino acids.

As used herein, the term “animal protein” refers to animal-based sources of protein. Such animal protein includes, for example and without any limitations, meat (for example, pork, beef, or veal), poultry (for example, chicken), fish, organs (for example, liver, spleen, or heart), viscera (for example, viscera of chicken or pork), and combinations thereof. As animal proteins, one can select, for example and without any limitation, animal proteins from poultry, beef, chicken, chicken meal, lamb, lamb meal, dried egg, fish, fish meal, meat and bone meal, meat byproducts, meat meal, turkey, blood plasma or bone marrow.

As used herein, and unless specified otherwise, the term “nucleotides” refers to 5′ nucleotides, which are nucleotides with a phosphoric acid group in the 5′-position of ribose. A “nucleotide” is understood to be a subunit of deoxyribonucleic acid (“DNA”) or ribonucleic acid (“RNA”). Such 5′nucleotides can, for example and without any limitation, be obtained through treatment of a starting material with 5′ nucleotidases and/or phosphatases. In certain embodiments, a 5′nucleotide encompassed by the present disclosure can be selected from the group consisting of adenosine monophosphate (AMP), guanosine monophosphate (GMP), inosine monophosphate (IMP), uridine monophosphate (UMP), cytidine monophosphate (CMP), thymidine monophosphate (TMP), xanthosine monophosphate (XMP), and a mixture of two or more thereof. For example, but without any limitation, the nucleotide can be AMP, GMP, IMP, or a mixture thereof. In certain exemplary embodiments, the nucleotide can be GMP alone, IMP alone, or a mixture thereof.

As used herein, and unless specified otherwise, the term “nucleotide source” refers to any source, in particular natural source, natural, processed, or provided as raw material which contains nucleotides as defined above. For example but without any limitation, a nucleotide source encompassed by the present disclosure can be a hydrolyzed nucleotide; e.g., partly or completely hydrolyzed nucleotide. In certain embodiments, a nucleotide source encompassed by the present disclosure can be a biological extract. In certain embodiments, the biological extract can be a bacterial extract or a yeast extract. In certain embodiments, the nucleotide source is a yeast extract. In certain embodiments, the yeast of the nucleotide source can be Kluyveromyces or Saccharomyces cerevisiae. In certain embodiments, the yeast of the nucleotide source can be Torula (Cyberlindnera jadinii). Further examples of nucleotide sources can include petMOD™ and/or petMOD™S feed material sources, which are commercialized by PROSOL S.p.A. Quality Control of Yeast Extract Nucleotides can be achieved by any method known in the art, such as ion-pair high-performance liquid chromatography.

As used herein, the term “derivative” refers to a compound that is derived from some other compound and maintains its general structure. For example, but without any limitation, trichloromethane (chloroform) is a derivative of methane.

The term “enantiomers” refers to a pair of stereoisomers that are non-superimposable mirror images of each other. A 1:1 mixture of a pair of enantiomers is a “racemic” mixture or a racemate. The term is used to designate a racemic mixture where appropriate.

The term “enantiopure” refers to a sample that within the limits of detection consists of a single enantiomer.

The term “diastereoisomers” refers to stereoisomers that have at least two asymmetric atoms, but which are not mirror-images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levo-rotatory) in which they rotate plane polarized light at the wavelength of the sodium D line.

The term “isomers” refers to different compounds that have the same molecular formula but differ in arrangement and configuration of the atoms. Also, as used herein, the term “stereoisomer” refers to any of the various stereo isomeric configurations which can exist for a given compound of the presently disclosed subject matter and includes geometric isomers. It is understood that a substituent can be attached at a chiral center of a carbon atom. Also, as used herein, the terms “constitutional isomers” refers to different compounds which have the same numbers of, and types of, atoms but the atoms are connected differently.

### 2. Flavor Compositions

In certain embodiments, the present disclosure provides flavor compositions with improved palatability. For example, without any limitation, the flavor composition disclosed herein can improve an umami taste.

In certain embodiments, the flavor composition binds to an umami receptor. Umami receptors (e.g., T1R1/T1R3) detect food molecules that can elicit taste qualities and properties, e.g., improved palatability or umami taste. The detection of the molecules eliciting taste qualities by the umami receptors improves the willingness of the animal, e.g., cat or dog, to eat a particular food. Additional information concerning the properties of the umami receptors can be found in International Patent Application Nos. PCT/EP2013/072794 and PCT/US2015/065046, which are incorporated by reference in their entirety. In certain embodiments, the umami receptor is a feline T1R1 protein. In certain embodiments, the T1R1 protein comprises or consists of the amino acid sequence set forth in SEQ ID NO: 1. In certain embodiments, the umami receptor is a feline T1R3 protein. In certain embodiments, the T1R3 protein comprises or consists of the amino acid sequence set forth in SEQ ID NO: 2. SEQ ID NOs: 1-2 are provided below.

(SEQ ID NO: 1)
MSLPAAHLVGLQLSLSCCWALSCHSTETSADFSLPGDYLLAGLFPLHSDCPGVRHRPTVT
LCDRPDSFNGHGYHLFQAMRFGIEEINNSTALLPNVTLGYQLYDVCSESANVYATLNVLS
LLGTHHVEIRADPSHYSPAALAVIGPDTTNHAATTAALLSPFLVPLISYEASSVTLGVKR
HYPSFLRTIPSDKHQVEAMVLLLQSFGWVWISVVGSDGDYGQLGVQALEEQATQQGICVA
FKDIIPFSARPGDERMQGIMHHLARARTTVVVVFSSRQLARVFFESVVLANLTAKVWIAS
EDWAISRHISNVPGIQGIGTVLGVAIQQRLVPGLKEFEEAYVQADKGAPGPCSRTSECSS
NQLCRECRAFTAEQMPTLGAFSMSSAYNAYRAVYAVAHGLHQLLGCASGACSRDRVYPWQ
LLEQIRKVNFLLHKDTVRFNDNGDPLSGYDIIAWDWSGPKWNFRVIGSSMWPPVQLDINK
TKIRWHGKDNQVPKSVCSSDCLEGHQRVISGFYHCCFECVPCEAGSFLNKSDLHSCQPCG
KEEWAPAGSETCFPRTVVFLTWHETISWVLLAANTLLLLLVTGTAGLFAWHLDTPVVKSA
GGRLCFFMLGSLAGGSCGLYGFFGEPTLPTCLLRQSLLALGFAIFLSCLTIRSFQLVFIF
KFSAKVPTFYRAWVQNHGPGLFVVISSMAQLLICLTWLAVWTPLPTREYQRFPQLVVLDC
TEANSPGFMLAFAYNGLLSVSAFACSYLGKDLPENYNEAKCVTFSLLLNFVSWIAFFTTA
SVYQGKYLPAVNVLAALSSLSGGFSGYFLPKCYVILCRPDLNSTEHFQASIQEYTRRCGS
T
(SEQ ID NO: 2)
MPGLALLGLTALLGLTALLDHGEGATSCLSQQLRMQGDYVLGGLFPLGSAEGTGLGDGLQ
PNATVCTRFSSLGLLWALAVKMAVEEINNGSALLPGLHLGYDLFDTCSEPMVAMKPSLVF
MAKAGSCSIAAYCNYTQYQPRVLAVIGPHSSELALVTGKFFSFFLVPQVSYGASTDRLSN
REIFPSFFRTVPSDQVQVAAMVELLQELGWNWVAAVGSDDEYGRQGLSLFSGLASARGIC
IAHEGLVPLPPGSLRLGALQGLLRQVNQSSVQVVVLFSSAHAARTLFSYSIRCKLSPKVW
VASEAWLTSDLVMTLPGMPGVGTVLGFLQQGAPMPEFPSYVRTRLALAADPAFCASLDAE
QPGLEEHVVGPRCPQCDHVTLENLSAGLLHHQTFAAYAAVYGVAQALHNTLRCNASGCPR
REPVRPWQLLENMYNVSFRARGLALQFDASGNVNVDYDLKLWVWQDPTPELRTVGTFKGR
LELWRSQMCWHTPGKQQPVSQCSRQCKEGQVRRVKGFHSCCYDCVDCKAGSYQRNPDDLL
CTQCDQDQWSPDRSTRCFARKPMFLAWGEPAVLLLLALLALALGLALAALGLFLWHSDSP
LVQASGGPRACFGLACLGLVCLSVLLFPGQPGPASCLAQQPLFHLPLTGCLSTLFLQAAE
IFVGSELPPSWAEKMRGRLRGPWAWLVVLLAMLAEAALCAWYLVAFPPEVVTDWRVLPTE
ALVHCHVHSWISFGLVHATNAMLAFLCFLGTFLVQSRPGRYNGARGLTFAMLAYFITWIS
FVPLFANVHVAYQPAVQMGTILLCALGILATFHLPKCYLLLQRPELNTPEFFLEDNARAQ
GSSWGQGRGESGQKQVTPDPVTSPQ

In certain embodiments, the presently disclosed flavor compositions include one or more amino acids or derivatives thereof. In certain embodiments, the flavor composition includes a first amino acid or a derivative thereof. In certain embodiments, the first amino acid is capable of activating an umami receptor. In certain embodiments, the first amino acid is an agonist of an umami receptor. In certain embodiments, the first amino acid is alanine, histidine, glycine, serine, tyrosine, or a mixture thereof. In certain embodiments, the first amino acid is histidine. In certain embodiments, the first amino acid is alanine. In certain embodiments, the first amino acid is glycine. In certain embodiments, the first amino acid is serine. In certain embodiments, the first amino acid is tyrosine.

In certain embodiments, the flavor composition includes a second amino acid or a derivative thereof. In certain embodiments, the second amino acid is not capable of activating an umami receptor. In certain embodiments, the second is an allosteric modulator of an umami receptor. In certain embodiments, the second amino acid is proline, hydroxyproline, valine, threonine, lysine, or a mixture thereof. In certain embodiments, the second amino acid is proline. In certain embodiments, the second amino acid is hydroxyproline. In certain embodiments, the second amino acid is valine. In certain embodiments, the second amino acid is threonine. In certain embodiments, the second amino acid is lysine.

In certain embodiments, the flavor composition includes a first amino acid and a second amino acid. In certain embodiments, the first amino acid is histidine and the second amino acid is proline. In certain embodiments, the first and second amino acid can be present at a ratio. In certain embodiments, the molar ratio of the first and second amino acids in the flavor composition can be between about 1:100 and about 1:1, between about 1:100 and about 1:2, between about 1:100 and about 1:5, between about 1:100 and about 1:10, between about 1:100 and about 1:20, between about 1:50 and about 1:1, between about 1:50 and about 1:2, between about 1:50 and about 1:5, or between about 1:50 and about 1:10, by mole. In certain embodiments, the ratio can be between about 1:30 and about 1:1, between about 1:30 and about 1:2, between about 1:30 and about 1:5, between about 1:30 and about 1:10, between about 1:20 and about 1:1, between about 1:20 and about 1:2, or between about 1:20 and about 1:5. In certain embodiments, the molar ratio can be between about 1:15 and about 1:1, between about 1:15 and about 1:2, between about 1:15 and about 1:5, between about 1:10 and about 1:1, between about 1:10 and about 1:2, between about 1:10 and about 1:5, between about 1:5 and about 1:1, or between about 1:5 and about 1:2, e.g., about 1:12 or about 1:6. In certain embodiments, the first and second amino acids can be present in a molar ratio of about 50:1, about 30:1, about 10:1, about 5:1, about 3:1, about 1:1, about 0.02:1, or about 0.015:1. In certain embodiments, the first and second amino acids can be present in a molar ratio of about 0.5:1, about 0.6:1, about 0.7:1, about 0.8:1, about 0.9:1, about 0.71:1, about 0.72:1, about 0.73:1, about 0.74:1, about 0.75:1, about 0.76:1, about 0.77:1, about 0.78:1, or about 0.79:1. In certain embodiments, the first and second amino acids can be present in a molar ratio of about 1:1.1, about 1:1.2, about 1:1.3, about 1:1.4, about 1:1.5, about 1:1.6, about 1:1.7, about 1:1.8, or about 1:1.9. In certain embodiments, the first and second amino acids can be present in a molar ratio of about 1:1. In certain embodiments, the first and second amino acids can be present in a molar ratio of about 1:1.3.

In certain embodiments, the flavor composition includes a milk protein, a derivative thereof, or a salt thereof. In certain embodiments, the milk protein is a casein or a salt thereof. Casein constitutes approximately 80% (29.5 g/L) of the total protein in milk. Casein is chiefly phosphate-conjugated and includes calcium phosphate-micelle complexes. Casein includes a heterogeneous family of 4 major components including alpha- (as₁- and as₂-casein), beta-, gamma-, and kappa-casein. In certain embodiments, the flavor composition includes casein. In certain embodiments, the flavor composition includes calcium caseinate. In certain embodiments, the flavor composition includes sodium caseinate. In certain embodiments, the milk protein comprises a casein hydrolysate. Casein hydrolysates are prepared by hydrolyzing a casein substrate (e.g., sodium caseinate). In certain embodiments, casein hydrolysates are prepared by enzyme hydrolysis. In certain embodiments, casein hydrolysates are prepared by acid hydrolysis. In certain embodiments, the flavor composition does not include a milk protein, a derivative thereof, or a salt thereof. In certain embodiments, the flavor composition includes a nucleotide. As used herein, the term “nucleotide” refers to an organic compound having a nitrogen-containing purine or pyrimidine base linked to a sugar (ribose or deoxyribose) and a phosphate group. Non-limiting examples of nucleotides include guanosine monophosphate (GMP), guanosine diphosphate (GDP), guanosine triphosphate (GTP), adenosine monophosphate (AMP), adenosine diphosphate (ADP), adenosine triphosphate (ATP), cytidine monophosphate (CMP), cytidine diphosphate (CDP), cytidine triphosphate (CTP), inosine monophosphate (IMP), inosine diphosphate (IDP, inosine triphosphate (ITP), uridine monophosphate (UMP), uridine diphosphate (UDP), uridine triphosphate (UTP), thymidine monophosphate (TMP), thymidine diphosphate (TDP), thymidine triphosphate (TTP), and xanthosine monophosphate (XMP), xanthosine diphosphate (XDP), and xanthosine triphosphate (XTP). In certain embodiments, the nucleotide is GMP. In certain embodiments, the nucleotide is obtained from a yeast extract. In certain embodiments, the yeast extract can be a Kluyveromyces yeast extract, a Torula yeast extract, or a Saccharomyces cerevisiae yeast extract. In certain embodiments, the flavor composition includes a nucleotide derivative. Additional information and examples of nucleotide derivatives encompassed by the present disclosure can be found in International Patent Application No. PCT/US2015/065067, which is incorporated by reference in its entirety. In certain embodiments, the flavor composition does not include a nucleotide or derivative thereof.

Additionally or alternatively, the flavor composition includes a yeast extract. In certain embodiments, the yeast extract can be obtained from different types of yeast. For example, but without any limitation, the yeast extract can be obtained from Saccharomyces, Pichia, Kluyveromyces, Hansenula, Candida, and Torula. In certain embodiments, the yeast extract is obtained from a Saccharomyces yeast. In certain embodiments, the yeast extract is obtained from a Kluyveromyces yeast. In certain embodiments, the yeast extract is obtained from a Torula yeast. In certain embodiments, the flavor composition includes a furan or a derivative thereof. Furan is a heterocyclic organic compound having a five-membered aromatic ring with four carbon atoms and one oxygen atom. Chemical compounds containing such rings are also referred to as furans. Non-limiting examples of furan derivatives include furaneol, methoxyfuraneol, furazolidone, nifuratel, and furaltadone. In certain embodiments, the flavor composition comprises furaneol. In certain embodiments, the flavor composition does not include a nucleotide or derivative thereof.

In certain embodiments, the flavor composition includes a phosphate. As used herein, a “phosphate” is a salt-based formally on phosphorus(V) oxoacids and in particular salt of phosphoric(V) acid, H₃PO₄. A large number of polymeric phosphates also exist containing P—O—P bridges. In certain embodiments, the phosphate can be a linear polyphosphate, a cyclic polyphosphate, a cross-linked polyphosphate, or an ultraphosphate. In certain embodiments, the phosphate is a pyrophosphate (PPi). In certain embodiments, the flavor composition does not include a nucleotide or derivative thereof.

In certain embodiments, the flavor composition includes a vitamin. Non-limiting examples of vitamins include vitamin A, vitamin C, vitamin D, vitamin, E, vitamin K, vitamin B1 (thiamine), vitamin B2 (riboflavin), vitamin B3 (niacin), vitamin B6 (pyridoxine), vitamin B12 (cyanocobalamin), pantothenic acid, biotin, folate, choline, and carnitine.

In certain embodiments, the flavor composition includes a mineral. For example, but without any limitation, the minerals encompassed by the present disclosure include calcium, phosphorus, potassium, sodium, chloride, magnesium, iron, zinc, iodine, chromium, copper, fluoride, molybdenum, manganese, and selenium. In certain embodiments, the mineral is calcium chloride.

In certain embodiments, the flavor composition includes a thickening agent. For example, but without any limitation, the thickening agents encompassed by the present disclosure include starches from corn, wheat, rice, potato, tapioca, and derivatives thereof. Additional examples of thickening agents encompassed by the present disclosure include carrageenan, xanthan, guar, locust bean, and carboxymethylcellulose. In certain embodiments, the thickening agent is xanthan.

In certain embodiments, the flavor composition includes an antioxidant. Non-limiting examples of antioxidants include beta-carotene, catechins, copper, cryptoxanthins, flavonoids, indoles, isoflavonoids, lignans, lutein, lycopene, and polyphenols.

In certain embodiments, the compounds of the flavor composition (e.g., first and second amino acids) can include stereoisomers, enantiomers, diastereomers, or racemates of the compounds and molecule disclosed herein. The compounds of the present disclosure can contain one or more asymmetric centers and can thus give rise to enantiomers, diastereomers, and other stereoisomeric forms that can be defined, in terms of absolute stereochemistry, as (R)- or (S)-. The present disclosure is meant to include all such possible isomers, including racemic mixtures, optically pure forms, and intermediate mixtures. Optically active (R)- and (S)-isomers can be prepared using chiral synthons or chiral reagents or resolved using conventional techniques. If the compound contains a double bond, the substituent can be E or Z configuration. If the compound contains a disubstituted cycloalkyl, the cycloalkyl substituent can have a cis- or trans-configuration. All tautomeric forms are also intended to be included.

In certain embodiments, the compounds of the flavor composition can include a salt form of the compound. For example, but without any limitation, salt forms include an acetate salt, a formate salt, a TFA salt, or a sulfonate salt. In certain embodiments, the compound salt comprises an anion (−) (for example, but not limited to, Cl—, O²⁻, CO₃²⁻, HCO³⁻, OH⁻, NO³⁻, PO₄³⁻, SO₄²⁻, CH3COO⁻, HCOO⁻ and C₂O₄²⁻) bonded via an ionic bond with a cation (+) (for example, but not limited to, Al³⁺, Ca²⁺, Na⁺, K⁺, Cu²⁺, H⁺, Fe³⁺, Mg²⁺, NH⁴⁺, and H₃O⁺). In certain embodiments, the compound salt comprises a cation (+) bonded via an ionic bond with an anion (−).

In certain embodiments, the compounds of the flavor composition can be generated using standard chemosynthesis processes. In certain embodiments, the chemosynthesis process provides a compound having a purity of at least 99.999%, or at least 99%, or at least 95%, or at least 90%, or at least 85 or at least 80%. In certain embodiments, the compounds can be prepared using standard hydrolysis processes such as those employing acids, enzymes, or a combination of acids and enzymes.

In certain embodiments, the compounds of the flavor composition can also be generated under food preparation conditions, e.g., during the production of a pet food product. For example, but not by way of limitation, the compounds of the present disclosure can be generated during a thermal food process, e.g., sterilization, retorting, and/or extrusion, from precursor compounds present in the pet food. In certain embodiments, the flavor composition can be admixed with or generated in a liquid (e.g., drinking water). Alternatively or additionally, the flavor composition can be admixed with or dissolved in a liquid (e.g., drinking water).

### 2.1. Exemplary Flavor Compositions

In certain embodiments, the flavor composition comprises a first amino acid capable of activating an umami receptor and a second amino acid not capable of activating an umami receptor. In certain embodiments, the first amino acid is histidine. In certain embodiments, the second amino acid is proline. In certain embodiments, the first and second amino acids are at a molar ratio of about 1:1. In certain embodiments, the flavor composition does not comprise a milk protein. In certain embodiments, the flavor composition does not comprise a phosphate or derivative thereof. In certain embodiments, the flavor composition does not comprise a furan or derivative thereof.

In certain embodiments, the flavor composition consists of a first amino acid capable of activating an umami receptor and a second amino acid not capable of activating an umami receptor. In certain embodiments, the first amino acid is histidine. In certain embodiments, the second amino acid is proline. In certain embodiments, the first and second amino acids are at a molar ratio of about 1:1.

In certain embodiments, the flavor composition consists of a first amino acid capable of activating an umami receptor, a second amino acid not capable of activating an umami receptor, a yeast extract, a mineral, and a phosphate. In certain embodiments, the first amino acid is histidine. In certain embodiments, the second amino acid is proline. In certain embodiments, the first and second amino acids are at a molar ratio of about 1:1. In certain embodiments, the mineral is calcium chloride. In certain embodiments, the phosphate is pyrophosphate.

In certain embodiments, the flavor composition consists of a first amino acid capable of activating an umami receptor, a second amino acid not capable of activating an umami receptor, a yeast extract, a mineral, a phosphate, and a thickening agent. In certain embodiments, the first amino acid is histidine. In certain embodiments, the second amino acid is proline. In certain embodiments, the first and second amino acids are at a molar ratio of about 1:1. In certain embodiments, the mineral is calcium chloride. In certain embodiments, the phosphate is pyrophosphate. In certain embodiments, the thickening agent is xanthan.

In certain embodiments, the flavor composition comprises a first amino acid capable of activating an umami receptor and a second amino acid not capable of activating an umami receptor. In certain embodiments, the first amino acid is tyrosine. In certain embodiments, the second amino acid is lysine. In certain embodiments, the first and second amino acids are at a molar ratio of about 1:1.3. In certain embodiments, the flavor composition does not comprise a milk protein. In certain embodiments, the flavor composition does not comprise a phosphate or derivative thereof. In certain embodiments, the flavor composition does not comprise a furan or derivative thereof.

In certain embodiments, the flavor composition comprises a first amino acid capable of activating an umami receptor and a second amino acid not capable of activating an umami receptor. In certain embodiments, the first amino acid is tyrosine. In certain embodiments, the second amino acid is lysine. In certain embodiments, the first and second amino acids are at a molar ratio of about 1:1.3.

In certain embodiments, the flavor composition comprises a first amino acid capable of activating an umami receptor, a second amino acid not capable of activating an umami receptor, and a yeast extract. In certain embodiments, the first amino acid is tyrosine. In certain embodiments, the second amino acid is lysine. In certain embodiments, the first and second amino acids are at a molar ratio of about 1:1.3.

In certain embodiments, the flavor composition comprises a milk protein and a nucleotide or derivative thereof. In certain embodiments, the milk protein is a casein hydrolysate. In certain embodiments, the nucleotide is GMP. In certain embodiments, the flavor composition further comprises a furan or derivative thereof. In certain embodiments, the furan or derivative thereof is furaneol. In certain embodiments, the flavor composition further comprises a phosphate. In certain embodiments, the phosphate is pyrophosphate.

In certain embodiments, the flavor composition comprises a milk protein and a nucleotide or derivative thereof. In certain embodiments, the milk protein is a casein hydrolysate. In certain embodiments, the nucleotide is IMP. In certain embodiments, the flavor composition further comprises a furan or derivative thereof. In certain embodiments, the furan or derivative thereof is furaneol. In certain embodiments, the flavor composition further comprises a phosphate. In certain embodiments, the phosphate is pyrophosphate.

In certain embodiments, the flavor composition comprises a milk protein and a nucleotide mixture. In certain embodiments, the milk protein is a casein hydrolysate. In certain embodiments, the nucleotide mixture comprises or consists of GMP and IMP. In certain embodiments, the flavor composition further comprises a furan or derivative thereof. In certain embodiments, the furan or derivative thereof is furaneol. In certain embodiments, the flavor composition further comprises a phosphate. In certain embodiments, the phosphate is pyrophosphate.

### 3. Pet Food Products

The present disclosure provides pet food products including flavor compositions disclosed herein (see Section 2). In certain embodiments, the flavor composition is directly added to a pet food product. The flavor composition of the present disclosure provides an unexpected taste and imparts, for example, an umami sensory experience. The flavor compositions disclosed herein can be added prior to, during, or after formulation, processing, or packaging of the pet food product.

Non-limiting examples of suitable pet food products include wet food products, dry food products, moist food products, pet food supplements (e.g., vitamins), pet beverage products, snack and treats, and pet food categories described herein.

In certain embodiments, the pet food product is a pet beverage product. In certain embodiments, the pet beverage product is drinking water. “Drinking water” is water that is safe to drink or use for food preparation. In certain embodiments, the drinking water can be tap water. In certain embodiments, the drinking water has a pH of between about 5.0 to about 8.0, from about 5.0 to about 7.5, from about 5.0 to about 7.0, from about 5.0 to about 6.5, from about 5.0 to about 6.0, from about 6.0 to about 8.0, from about 6.0 to about 7.5, from about 6.0 to about 7.0, from about 6.0 to about 6.9, from about 6.0 to about 6.8, from about 6.0 to about 6.7, from about 6.0 to about 6.6, from about 6.0 to about 6.5, from about 6.1 to about 6.5, from about 6.2 to about 6.5, from about 6.3 to about 6.5, from about 6.4 to about 6.5, from about 6.2 to about 6.5, from about 6.2 to about 6.4, from about 7.0 to about 8.0, from about 7.0 to about 7.9, from about 7.0 to about 7.8, from about 7.0 to about 7.7, from about 7.0 to about 7.6, from about 7.0 to about 7.5, from about 7.1 to about 7.5, from about 7.2 to about 7.5, from about 7.3 to about 7.5, from about 7.4 to about 7.5, from about 7.2 to about 7.5, or from about 7.2 to about 7.4.

In certain embodiments, the drinking water has a pH of from about 7.0 to about 7.1. In certain embodiments, the drinking water has a pH of about 7.03. In certain embodiments, the drinking water has a pH of about 7.13.

In certain embodiments, the drinking water has a pH of from about 6.8 to about 7.0. In certain embodiments, the drinking water has a pH of about 6.87. In certain embodiments, the drinking water has a pH of about 6.86. In certain embodiments, the drinking water has a pH of about 6.90.

In certain embodiments, the drinking water has a pH of from about 7.5 to about 7.8. In certain embodiments, the drinking water has a pH of about 7.79. In certain embodiments, the drinking water has a pH of about 7.77. In certain embodiments, the drinking water has a pH of about 7.75. In certain embodiments, the drinking water has a pH of about 7.74.

In certain embodiments, the flavor composition of the present disclosure is present in a pet food product in an amount that is sufficient to modulate, activate and/or enhance an umami receptor. For example, but not by way of limitation, a flavor composition can be present in the pet food product in an amount from about 1 nM to about 1 M, from about 1 μM to about 1 M, from about 1 mM to about 1 M, from about 10 mM to about 1 M, from about 100 mM to about 1 M, from about 250 mM to about 1 M, from about 500 mM to about 1 M, from about 750 mM to about 1 M, from about 0.001 μM to about 1 M, from about 0.001 μM to about 750 mM, from about 0.001 μM to about 500 mM, from about 0.001 M to about 250 mM, from about 0.001 μM to about 100 mM, from about 0.001 μM to about 50 mM, from about 0.001 μM to about 25 mM, from about 0.001 μM to about 10 mM, from about 0.001 μM to about 1 mM, from about 0.001 μM to about 100 μM or from about 0.001 μM to about 10 μM, and values in between.

In certain embodiments, the flavor composition is present in the pet food product in an amount of from about 0.001 ppm to about 10,000 ppm. For example, but not by way of limitation, the flavor composition can be present in the amount from about 0.001 ppm to about 7,500 ppm, from about 0.001 ppm to about 5,000 ppm, from about 0.001 ppm to about 2,500 ppm, from about 0.001 ppm to about 1,000 ppm, from about 0.001 ppm to about 750 ppm, from about 0.001 ppm to about 500 ppm, from about 0.001 ppm to about 250 ppm, from about 0.001 ppm to about 150 ppm, from about 0.001 ppm to about 100 ppm, from about 0.001 ppm to about 75 ppm, from about 0.001 ppm to about 50 ppm, from about 0.001 ppm to about 25 ppm, from about 0.001 ppm to about 15 ppm, from about 0.001 ppm to about 10 ppm, from about 0.001 ppm to about 5 ppm, from about 0.001 ppm to about 4 ppm, from about 0.001 ppm to about 3 ppm, from about 0.001 ppm to about 2 ppm, from about 0.001 ppm to about 1 ppm, from about 0.01 ppm to about 10,000 ppm, from about 0.01 ppm to about 7,500 ppm, from about 0.01 ppm to about 5,000 ppm, from about 0.01 ppm to about 2,500 ppm, from about 0.1 ppm to 1,000 ppm, from about 1 ppm to 1,000 ppm, from about 2 ppm to about 1,000 ppm, from about 3 ppm to about 1,000 ppm, from about 4 ppm to about 1,000 ppm, from about 5 ppm to about 1,000 ppm, from about 10 ppm to about 1,000 ppm, from about 15 ppm to about 1,000 ppm, from about 25 ppm to about 1,000 ppm, from about 50 ppm to about 1,000 ppm, from about 75 ppm to about 1,000 ppm, from about 100 ppm to about 1,000 ppm, from about 150 ppm to about 1,000 ppm, from about 250 ppm to about 1,000 ppm, from about 250 ppm to about 1,000 ppm, from about 500 ppm to about 1,000 ppm or from about 750 ppm to about 1,000 ppm, and values in between.

In certain embodiments, the flavor composition is present in the pet food product at an amount greater than about 0.001 ppm, greater than about 0.01 ppm, greater than about 0.1 ppm, greater than about 1 ppm, greater than about 2 ppm, greater than about 3 ppm, greater than about 4 ppm, greater than about 5 ppm, greater than about 10 ppm, greater than about 25 ppm, greater than about 50 ppm, greater than about 75 ppm, greater than about 100 ppm, greater than about 250 ppm, greater than about 500 ppm, greater than about 750 ppm, greater than about 1,000 ppm, greater than about 2,500 ppm, greater than about 5,000 ppm, greater than about 7,500 ppm, or greater than about 10,000 ppm, and values in between.

In certain embodiments, the flavor composition is admixed with the food product (e.g. drinking water) wherein the flavor composition is present in an amount of from about 0.0001 to about 10% weight/weight (w/w) of the food product. For example, but not by way of limitation, the flavor composition can be present in the amount from about 0.0001% to about 10%, from about 0.0001% to about 1%, from about 0.0001% to about 0.1%, from about 0.0001 to about 0.01%, from about 0.0001% to about 0.001%, from about 0.001% to about 10%, from about 0.001% to about 1%, from about 0.01% to about 1% or from about 0.1% to about 1%, and values in between.

In certain embodiments, where the flavor composition includes an amino acid or derivative thereof, the amino acid or derivative thereof can be present in the pet food product in an amount of from about 1 mM to about 1 M, or from about 250 mM to about 1 M, or from about 5 mM to about 500 mM, or from about 10 mM to about 100 mM, or from about 15 mM to about 50 mM, or from about 20 mM to about 40 mM of the pet food product. In certain embodiments, the amino acid or derivative thereof can be present at an amount less than about 1 M, less than about 200 mM, less than about 100 mM, less than about 50 mM, less than about 20 mM or less than about 10 mM of the pet food product. In certain embodiments, the amino acid or derivative thereof can be present in an amount of about 200 mM of the pet food product. In certain embodiments, the amino acid or derivative thereof can be present in an amount of about 100 mM of the pet food product.

In certain embodiments, where the flavor composition includes a milk protein, derivative thereof, or salt thereof, the milk protein, derivative thereof, or salt thereof can be present in the pet food product in an amount of from about 0.0001 to about 10% weight/weight (w/w) of the food product. In certain embodiments, the milk protein, derivative thereof, or salt thereof can be present in the amount from about 0.0001% w/w to about 10% w/w, from about 0.0001% w/w to about 1% w/w, from about 0.0001% w/w to about 0.1% w/w, from about 0.0001% w/w to about 0.01% w/w, from about 0.0001% w/w to about 0.001% w/w, from about 0.001% w/w to about 10% w/w, from about 0.001% w/w to about 1% w/w, from about 0.01% w/w to about 1% w/w, or from about 0.1% w/w to about 1% w/w, and values in between. In certain embodiments, the milk protein, derivative thereof, or salt thereof is present in the pet food product in an amount of from about 1% w/w to about 5% w/w, from about 1.5% w/w to about 5% w/w, from about 2% w/w to about 5% w/w, from about 2.5% w/w to about 5% w/w, from about 3% w/w to about 5% w/w, from about 3.5% to about 5% w/w, from about 4% to about 5% w/w, from about 4.5% w/w to about 5% w/w, and values in between. In certain embodiments, the milk protein, derivative thereof, or salt thereof is present in the pet food product in an amount of from about 1% w/w to about 5% w/w, from about 1% w/w to about 4.5% w/w, from about 1% w/w to about 4% w/w, from about 1% w/w to about 3.5% w/w, from about 1% w/w to about 3% w/w, from about 1% w/w to about 2.5% w/w, from about 1% w/w to about 2% w/w, from about 1% w/w to about 1.5% w/w, from about 2% w/w to about 4% w/w, from about 2.5% w/w to about 3.5% w/w, and values in between. In certain embodiments, the milk protein, derivative thereof, or salt thereof is present in an amount of about 3% w/w.

In certain embodiments, where the flavor composition includes a milk protein, derivative thereof, or salt thereof, the milk protein, derivative thereof, or salt thereof can be present in the pet food product in an amount of from about 0.0001 to about 10% weight/volume (w/v) of the food product. In certain embodiments, the milk protein, derivative thereof, or salt thereof can be present in the amount from about 0.0001% w/v to about 10% w/v, from about 0.0001% w/v to about 1% w/v, from about 0.0001% w/v to about 0.1% w/v, from about 0.0001% w/v to about 0.01% w/v, from about 0.0001% w/v to about 0.001% w/v, from about 0.001% w/v to about 10% w/v, from about 0.001% w/v to about 1% w/v, from about 0.01% w/v to about 1% w/v, or from about 0.1% w/v to about 1% w/v, and values in between. In certain embodiments, the milk protein, derivative thereof, or salt thereof is present in the pet food product in an amount of from about 1% w/v to about 5% w/v, from about 1.5% w/v to about 5% w/v, from about 2% w/v to about 5% w/v, from about 2.5% w/v to about 5% w/v, from about 3% w/v to about 5% w/v, from about 3.5% to about 5% w/v, from about 4% to about 5% w/v, from about 4.5% w/v to about 5% w/v, and values in between. In certain embodiments, the milk protein, derivative thereof, or salt thereof is present in the pet food product in an amount of from about 1% w/v to about 5% w/v, from about 1% w/v to about 4.5% w/v, from about 1% w/v to about 4% w/v, from about 1% w/v to about 3.5% w/v, from about 1% w/v to about 3% w/v, from about 1% w/v to about 2.5% w/v, from about 1% w/v to about 2% w/v, from about 1% w/v to about 1.5% w/v, from about 2% w/v to about 4% w/v, from about 2.5% w/v to about 3.5% w/v, and values in between. In certain embodiments, the milk protein, derivative thereof, or salt thereof is present in an amount of about 3% w/v.

In certain embodiments, where the flavor composition includes a nucleotide or derivative thereof, the nucleotide or derivative thereof can be present in the pet food product in an amount of from about 1 mM to about 1 M, or from about 250 mM to about 1 M, or from about 5 mM to about 500 mM, or from about 10 mM to about 100 mM, or from about 15 mM to about 50 mM, or from about 20 mM to about 40 mM of the pet food product. In certain embodiments, the nucleotide or derivative thereof can be present at an amount less than about 1 M, less than about 200 mM, less than about 100 mM, less than about 50 mM, less than about 20 mM or less than about 10 mM of the pet food product. In certain embodiments, the nucleotide or derivative thereof can be present in an amount of about 5 mM of the pet food product.

In certain embodiments, where the flavor composition includes a furan or derivative thereof, the nucleotide or derivative thereof can be present in the pet food product in an amount greater than about 0.001 ppm, greater than about 0.01 ppm, greater than about 0.1 ppm, greater than about 1 ppm, greater than about 2 ppm, greater than about 3 ppm, greater than about 4 ppm, greater than about 5 ppm, greater than about 10 ppm, greater than about 25 ppm, greater than about 50 ppm, greater than about 75 ppm, greater than about 100 ppm, greater than about 250 ppm, greater than about 500 ppm, greater than about 750 ppm, greater than about 1,000 ppm, greater than about 2,500 ppm, greater than about 5,000 ppm, greater than about 7,500 ppm, greater than about 10,000 ppm, and values in between. In certain embodiments, the furan or derivative thereof can be present in an amount of about 4 ppm of the pet food product.

In certain embodiments, where the flavor composition includes a phosphate, the phosphate can be present in the pet food product in an amount of from about 1 mM to about 1 M, or from about 250 mM to about 1 M, or from about 5 mM to about 500 mM, or from about 10 mM to about 100 mM, or from about 15 mM to about 50 mM, or from about 20 mM to about 40 mM of the pet food product. In certain embodiments, the phosphate can be present at an amount less than about 1 M, less than about 200 mM, less than about 100 mM, less than about 50 mM, less than about 20 mM or less than about 10 mM of the pet food product. In certain embodiments, the phosphate can be present in an amount of about 10 mM of the pet food product.

In certain embodiments, where the flavor composition includes calcium chloride, the calcium chloride can be present in the pet food product in an amount of from about 1 mM to about 1 M, or from about 250 mM to about 1 M, or from about 5 mM to about 500 mM, or from about 10 mM to about 100 mM, or from about 15 mM to about 50 mM, or from about 20 mM to about 40 mM of the pet food product. In certain embodiments, the calcium chloride can be present in the pet food product in an amount of from about 1 mM to about 20 mM, from about 5 mM to about 20 mM, or from about 10 mM to about 20 mM, or from about 10 mM to about 15 mM, or from about 5 mM to about 15 mM, or from about 5 mM to about 10 mM of the pet food product. In certain embodiments, the phosphate can be present at an amount less than about 1 M, less than about 200 mM, less than about 100 mM, less than about 50 mM, less than about 20 mM or less than about 10 mM of the pet food product. In certain embodiments, the phosphate can be present in an amount of from about 5 mM to about 15 mM of the pet food product.

In certain embodiments, where the flavor composition includes xanthan, the xanthan can be present in the pet food product in an amount of from about 1 g/L to about 10 g/L, or from about 1 g/L to about 9 g/L, or from about 1 g/L to about 8 g/L, or from about 1 g/L to about 7 g/L, or from about 1 g/L to about 6 g/L, or from about 1 g/L to about 5 g/L, or from about 2 g/L to about 5 g/L, or from about 2 g/L to about 6 g/L, or from about 2 g/L to about 7 g/L, or from about 2 g/L to about 8 g/L, or from about 2 g/L to about 9 g/L, or from about 3 g/L to about 4 g/L, or from about 3 g/L to about 5 g/L, or from about 3 g/L to about 6 g/L, or from about 3 g/L to about 7 g/L of the pet food product. In certain embodiments, the phosphate can be present at an amount less than about 10 g/L, less than about 9 g/L, less than about 8 g/L, less than about 7 g/L, less than about 6 g/L or less than about 5 g/L of the pet food product. In certain embodiments, the phosphate can be present in an amount of from about 3 g/L to about 7 g/L of the pet food product.

In certain embodiments, the present disclosure relates to methods for increasing the taste of a pet food product comprising a) providing at least one pet food product, or a precursor thereof, and b) combining the pet food product (e.g. drinking water) with at least a flavor composition, for example, comprising a first and second amino acid, so as to form an enhanced pet food product. In certain embodiments, the flavor composition comprises an allosteric modulator, for example, a positive allosteric modulator.

### 3.1. Exemplary Pet Food Products

In certain embodiments, the pet food product is a drinking water comprising a flavor composition comprising a first amino acid capable of activating an umami receptor and a second amino acid not capable of activating an umami receptor. In certain embodiments, the first amino acid is histidine. In certain embodiments, the second amino acid is proline. In certain embodiments, the first and second amino acids are at a molar ratio of about 1:1. In certain embodiments, the first amino acid is present in an amount of about 100 mM of the pet food product. In certain embodiments, the second amino acid is present in an amount of about 100 mM of the pet food product. In certain embodiments, the pet food product does not comprise a milk protein. In certain embodiments, the pet food product does not comprise a phosphate or derivative thereof. In certain embodiments, the pet food product does not comprise a furan or derivative thereof.

In certain embodiments, the pet food product is a drinking water comprising a flavor composition consisting of a first amino acid capable of activating an umami receptor and a second amino acid not capable of activating an umami receptor. In certain embodiments, the first amino acid is histidine. In certain embodiments, the second amino acid is proline. In certain embodiments, the first and second amino acids are at a molar ratio of about 1:1. In certain embodiments, the first amino acid is present in an amount of about 100 mM of the pet food product. In certain embodiments, the second amino acid is present in an amount of about 100 mM of the pet food product.

In certain embodiments, the pet food product is a drinking water comprising a flavor composition consisting of a first amino acid capable of activating an umami receptor, a second amino acid not capable of activating an umami receptor, a yeast extract, a mineral, and a phosphate. In certain embodiments, the first amino acid is histidine. In certain embodiments, the second amino acid is proline. In certain embodiments, the first and second amino acids are at a molar ratio of about 1:1. In certain embodiments, the first amino acid is present in an amount of about 100 mM of the pet food product. In certain embodiments, the second amino acid is present in an amount of about 100 mM of the pet food product. In certain embodiments, the mineral is calcium chloride. In certain embodiments, the phosphate is pyrophosphate.

In certain embodiments, the pet food product is a drinking water comprising a flavor composition consisting of a first amino acid capable of activating an umami receptor, a second amino acid not capable of activating an umami receptor, a yeast extract, a mineral, a phosphate, and a thickening agent. In certain embodiments, the first amino acid is histidine. In certain embodiments, the second amino acid is proline. In certain embodiments, the first and second amino acids are at a molar ratio of about 1:1. In certain embodiments, the first amino acid is present in an amount of about 100 mM of the pet food product. In certain embodiments, the second amino acid is present in an amount of about 100 mM of the pet food product. In certain embodiments, the mineral is calcium chloride. In certain embodiments, the phosphate is pyrophosphate. In certain embodiments, the thickening agent is xanthan.

In certain embodiments, the pet food product is a drinking water comprising a flavor composition comprising a first amino acid capable of activating an umami receptor and a second amino acid not capable of activating an umami receptor. In certain embodiments, the first amino acid is tyrosine. In certain embodiments, the second amino acid is lysine. In certain embodiments, the first and second amino acids are at a molar ratio of about 1:1.3. In certain embodiments, the first amino acid is present in an amount of from about 20 mM to about 30 mM of the pet food product. In certain embodiments, the second amino acid is present in an amount of from about 20 mM to about 30 mM of the pet food product. In certain embodiments, the pet food product does not comprise a milk protein. In certain embodiments, the pet food product does not comprise a phosphate or derivative thereof. In certain embodiments, the pet food product does not comprise a furan or derivative thereof.

In certain embodiments, the pet food product is a drinking water comprising a flavor composition comprising a first amino acid capable of activating an umami receptor and a second amino acid not capable of activating an umami receptor. In certain embodiments, the first amino acid is tyrosine. In certain embodiments, the second amino acid is lysine. In certain embodiments, the first and second amino acids are at a molar ratio of about 1:1.3. In certain embodiments, the first amino acid is present in an amount of from about 20 mM to about 30 mM of the pet food product. In certain embodiments, the second amino acid is present in an amount of from about 20 mM to about 30 mM of the pet food product.

In certain embodiments, the pet food product is a drinking water comprising a flavor composition comprising a first amino acid capable of activating an umami receptor, a second amino acid not capable of activating an umami receptor, and a yeast extract. In certain embodiments, the first amino acid is tyrosine. In certain embodiments, the second amino acid is lysine. In certain embodiments, the first and second amino acids are at a molar ratio of about 1:1.3. In certain embodiments, the first amino acid is present in an amount of from about 20 mM to about 30 mM of the pet food product. In certain embodiments, the second amino acid is present in an amount of from about 20 mM to about 30 mM of the pet food product.

In certain embodiments, the pet food product is a drinking water comprising a flavor composition comprising a milk protein and a nucleotide. In certain embodiments, the milk protein is a casein hydrolysate. In certain embodiments, the casein hydrolysate is present in an amount of about 3%. In certain embodiments, the nucleotide is GMP. In certain embodiments, the GMP is present in an amount of about 10 mM of the pet food product. In certain embodiments, the flavor composition further comprises a furan or derivative thereof. In certain embodiments, the furan or derivative thereof is furaneol. In certain embodiments, the furaneol is present in an amount of about 4 ppm of the pet food product. In certain embodiments, the flavor composition further comprises a phosphate. In certain embodiments, the phosphate is pyrophosphate. In certain embodiments, the phosphate is present in an amount of about 10 mM of the pet food product.

In certain embodiments, the pet food product is a drinking water comprising a flavor composition comprising a milk protein and a nucleotide mixture. In certain embodiments, the milk protein is a casein hydrolysate. In certain embodiments, the casein hydrolysate is present in an amount of about 3%. In certain embodiments, the nucleotide mixture comprises or consists of GMP and IMP. In certain embodiments, the nucleotide mixture is present in an amount of about 10 mM of the pet food product. In certain embodiments, the flavor composition further comprises a furan or derivative thereof. In certain embodiments, the furan or derivative thereof is furaneol. In certain embodiments, the furaneol is present in an amount of about 4 ppm of the pet food product. In certain embodiments, the flavor composition further comprises a phosphate. In certain embodiments, the phosphate is pyrophosphate. In certain embodiments, the phosphate is present in an amount of about 10 mM of the pet food product.

In certain embodiments, the pet food product is a drinking water comprising a flavor composition comprising a milk protein and a nucleotide. In certain embodiments, the milk protein is a casein hydrolysate. In certain embodiments, the casein hydrolysate is present in an amount of about 3%. In certain embodiments, the nucleotide is IMP. In certain embodiments, the IMP is present in an amount of about 10 mM of the pet food product. In certain embodiments, the flavor composition further comprises a furan or derivative thereof. In certain embodiments, the furan or derivative thereof is furaneol. In certain embodiments, the furaneol is present in an amount of about 4 ppm of the pet food product. In certain embodiments, the flavor composition further comprises a phosphate. In certain embodiments, the phosphate is pyrophosphate. In certain embodiments, the phosphate is present in an amount of about 10 mM of the pet food product.

### 4. Formulations and Delivery Systems

In certain embodiments, the flavor compositions or the pet food products disclosed herein can be included in a package. As used herein, the term “package” refers to one or more containers and is considered a unit for manufacture, distribution, sale, or use. In certain non-limiting embodiments, a package includes a bag, a box, a carton, a bottle, a package of any type or design or material, over-wrap, shrink-wrap, affixed components (e.g., stapled, adhered, or the like), or combinations thereof. In certain embodiments, a package can include a drinking water comprising a flavor composition disclosed herein which is considered a unit for manufacture, distribution, sale, or use.

In certain embodiments, the present disclosure provides for effervescent composition including flavor compositions disclosed herein. Effervescent compositions are based on the reaction of an acid and a carbonate salt to form carbon dioxide and is known for being self-dissolving upon addition to water. In certain embodiments, the effervescent composition includes an effervescent agent. In certain embodiments, the effervescent agent includes an acid and a carbonate base. For example, without any limitation, the effervescent agent can include phosphoric acid, citric acid, malic acid, tartaric acid, adipic acid, fumaric acid, sodium bicarbonate, potassium bicarbonate, sodium carbonate, potassium carbonate, or a combination thereof.

In certain embodiments, the effervescent composition can be in the form of a tablet or powder. Tablet form can be particularly useful because conveniently packaged in a tube or a foil or blister packet enabling convenient storage and portability by the user. In certain embodiments, the effervescent composition can include additional components such as binders (e.g., dextrose or lactose, or sorbitol) or lubricants (e.g., polyethylene glycol, adipic acid, or sodium benzoate).

In certain embodiments, the flavor compositions of the present application can be incorporated into a delivery system for use in pet food products. Delivery systems can be liquid or solid, aqueous or non-aqueous. Delivery systems are generally adapted to suit the needs of flavor composition and/or the pet food product into which the flavor composition will be incorporated.

In certain embodiments, the flavor compositions can be employed in liquid form, dried form and/or solid form. When used in dried form, suitable drying means such as spray drying can be used. Alternatively, a flavor composition can be encapsulated or absorbed onto water-soluble materials, including but not limited to materials such as cellulose, starch, sugar, maltodextrin, gum arabic and so forth. The actual techniques for preparing such dried forms are well-known in the art and can be applied to the presently disclosed subject matter.

In certain embodiments, the flavor compositions can be used in many distinct physical forms well known in the art to provide an initial burst of taste, flavor, and/or texture; and/or a prolonged sensation of taste, flavor, and/or texture. Without being limited thereto, such physical forms include free forms, such as spray dried, powdered, and beaded forms and encapsulated forms, and mixtures thereof.

In certain embodiments, the flavor composition can be fully or partially encapsulated. Encapsulating materials and/or techniques can be selected to determine the type of modification of the flavor system. In certain embodiments, the encapsulating materials and/or techniques are selected to improve the stability of the flavor compositions. In certain embodiments, the encapsulating materials and/or techniques are selected to modify the release profile of the flavor compositions. Suitable encapsulating materials can include, but are not limited to, hydrocolloids such as alginates, pectins, agars, guar gums, celluloses, and the like, proteins, polyvinyl acetate, polyethylene, crosslinked polyvinyl pyrrolidone, polymethylmethacrylate, polylactidacid, polyhydroxyalkanoates, ethylcellulose, polyvinyl acetatephthalate, polyethylene glycol esters, methacrylicacid-co-methylmethacrylate, ethylene-vinylacetate (EVA) copolymer, and the like, and combinations thereof. Suitable encapsulating techniques can include, but are not limited to, spray coating, spray drying, spray chilling, absorption, adsorption, inclusion complexing (e.g., creating a flavor/cyclodextrin complex), coacervation, fluidized bed coating or other processes that can be used to encapsulate an ingredient with an encapsulating material. In certain embodiments, encapsulated delivery systems for flavor compositions can contain a hydrophobic matrix of fat or wax surrounding a sweetening agent or flavoring agent core. The fats can be selected from any number of conventional materials such as fatty acids, glycerides or polyglycerol esters, sorbitol esters, and mixtures thereof. Examples of fatty acids include but are not limited to hydrogenated and partially hydrogenated vegetable oils such as palm oil, palm kernel oil, peanut oil, rapeseed oil, rice bran oil, soybean oil, cottonseed oil, sunflower oil, safflower oil, and combinations thereof. Examples of glycerides include, but are not limited to, monoglycerides, diglycerides, and triglycerides.

Waxes can be chosen from the group consisting of natural and synthetic waxes and mixtures thereof. Non-limiting examples include paraffin wax, petrolatum, carbowax, microcrystalline wax, beeswax, carnauba wax, candellila wax, lanolin, bayberry wax, sugarcane wax, spermaceti wax, rice bran wax, and mixtures thereof.

The fats and waxes can be used individually or in combination in amounts varying from about 10 to about 70%, and alternatively in amounts from about 30 to about 60%, by weight of the encapsulated system. When used in combination, the fat and wax can be present in a ratio from about 70:10 to 85:15, respectively.

Typical encapsulated flavor compositions are disclosed in U.S. Pat. Nos. 4,597,970 and 4,722,845, the disclosures of which are incorporated herein by reference in their entireties.

In certain embodiments, the flavor composition is included in a liquid delivery system. In certain non-limiting embodiments, liquid delivery systems include systems with a dispersion of the flavor compositions disclosed herein, such as in carbohydrate syrups and/or emulsions.

Additionally or alternatively, solid delivery systems can be used. Solid delivery systems can be created by spray drying, spray coating, spray chilling, fluidized bed drying, absorption, adsorption, coacervation, complexation, or any other standard technique. In certain embodiments, the delivery system can be selected to be compatible with or to function in an edible composition. In certain embodiments, the delivery system includes an oleaginous material such as a fat or oil. In certain embodiments, the delivery system includes a confectionery fat such as cocoa butter, a cocoa butter replacer, a cocoa butter substitute, or a cocoa butter equivalent. When used in dried form, suitable drying means such as spray drying can be used. Alternatively, a flavor composition can be adsorbed or absorbed onto substrates such as water-soluble materials, such as cellulose, starch, sugar, maltodextrin, gum arabic and so forth can be encapsulated. The actual techniques for preparing such dried forms are well known in the art.

### 5. Methods of Treatment

The present disclosure provides for methods of preventing, treating, and/or delaying progression of chronic kidney disease (CKD) in an animal in need thereof. Furthermore, the present disclosure provides for methods of preventing, treating, and/or delaying dehydration associated with chronic kidney disease (CKD) in an animal in need thereof. In certain embodiments, the method comprises administering a flavor composition or a pet food product disclosed herein. Chronic Kidney Disease (CKD) is a common clinical condition in cats across all ages, although it is primarily diagnosed in cats over the age of 10 and can have a prevalence of 30-40% in older cats (Sparkes et al., Journal of feline medicine and surgery, 18(3), pp.219-239, 2016). This condition is characterized by a progressive decline in renal function resulting in increased retention of phosphorus (P) and protein catabolites (McCarron, Journal of the American Society of Nephrology 16: S93-S94, 2005). During early CKD, this condition is compensated for by increased glomerular pressure and filtration rate (Ames et al., J Vet Intern Med; 33:363-382, 2019), while calcium (Ca) and P balance is maintained through the stimulation of regulatory hormones fibroblast growth factor 23 (FGF-23) and parathyroid hormone (PTH) (Clarke, Endocrinology 152:4016-4018, 2011; Alexander et al., Br J Nutr; 121:1-21, 2019). However, over the long term, the above compensatory mechanisms can become maladaptive, contributing to progressive renal damage. Dietary management of cats with CKD aims to ameliorate the progression of the disease and improve the quality of life by correcting biochemical and mineral imbalances. The main feature of renal diets is a reduction of protein and phosphorus contents, alongside an increased caloric density and a neutral effect on acid-base balance. Renal diets contain higher calcium to phosphorus ratio (>1.5:1) in relation to maintenance diets (<1.5:1). Studies conducted in cats with early stage CKD (IRIS 1 and 2) have provided clinical evidence on optimal levels of protein, P, Ca, and Ca:P ratio for the long-term management of cats at early stages of the disease based on renal health parameters, Ca—P status, and mineral precipitation in the urinary tract of cats over a feeding period of 43 months (Schauf et al., Journal of Veterinary Internal Medicine 35.6 (2021): 2797-2811). The results of these studies indicated that feeding a moderately protein-restricted and P-restricted diet on a mixed dry-wet regimen (each format providing approximately 50% of the maintenance energy requirement) results in more stable blood creatinine, urea, and FGF23 over the long-term, while controlling Ca and P balance in cats with concomitant hypercalcemia.

The International Renal Interest Society (IRIS) recommends feeding a veterinary therapeutic renal diet for cats with CKD at stages 2, 3, and 4, which refers to cats with high levels of urea nitrogen and creatinine in blood (azotemic) with or without concomitant proteinuria and hypertension. This recommendation is supported by a number of clinical trials in cats demonstrating an efficacy of renal diets in reducing the frequency of uremic crisis (Harte et al., The Journal of nutrition 124: 2660S-2662S, 1994; Ross et al., Journal of the American Veterinary Medical Association 229: 949-957, 2006) and in increasing median survival rate (Elliott et al., The Journal of small animal practice 41: 235-242, 2000; Ross et al., Journal of the American Veterinary Medical Association 229: 949-957, 2006). With the progression of CKD (generally at IRIS 3 and 4), the ability of cats to concentrate their urine also starts to decline and cats will eventually start to drink more (polydipsia). In this scenario, cats are more vulnerable to dehydration, which can ultimately worsen renal function and can therefore benefit from feeding a moist diet, whilst having easy and plentiful access to water. Because of this, hydration therapy (including subcutaneous fluid administration) is commonly applied to cats with advanced stages of CKD. However, there is very little information currently on the beneficial effect of increasing water intake in cats at earlier stages of CKD (IRIS 1 and 2), which is the gap this project aims to close. In human studies it has been shown that increasing water intake has a beneficial effect on renal function in patients with all forms of CKD and in those at risk of developing CKD, meaning that increased water intake appears to have a preventative function in addition to helping with the symptoms of the disease once the symptoms appear (Clark et al., American journal of nephrology, 43(4), pp. 281-292, 2016).

The present disclosure also provides for methods of preventing, treating, and/or delaying progression of urinary tract disease in an animal in need thereof. In certain embodiments, the method comprises administering a flavor composition or a pet food product disclosed herein. As used herein, the term “urinary tract disease” refers to a range of disorders from mild to serious that occur in the urinary tract (e.g., lower urinary tract) and that include abnormalities in the structure and function of the bladder and the urethra. Most common clinical signs of urinary tract disease include, for example and without any limitation, difficult or painful urination, increased frequency of urination, crying out while urinating, blood in the urine, inappropriate urination (e.g., outside of the litter box), or frequent licking of the genital region (Dorsch et al., Journal of Feline Medicine and Surgery 21, no. 11 (November 2019): 1023-38; Hostutler et al., Veterinary Clinics: Small Animal Practice 35, no. 1 (2005): 147-170.).

In cats, urinary tract disease occurs mostly in middle-aged, over-weight cats that get little exercise, use an indoor litter box, have restricted access outdoor environments, and/or drink less water. Furthermore, stressful environmental factors such as living in a multi-cat household and changes in routine can increase the risk that a cat will develop urinary tract disease. Usually, diagnosis of urinary tract disease is based on clinical signs (e.g., physical examination and urinalysis) and laboratory-based methods (e.g., blood work, x-rays, abdominal ultrasound, and/or urine culture). The etiopathogenesis of urinary tract disease include multiple causes such as infections, inflammation, diet, and behavioral issues. For example, but without any limitation, the causes of urinary tract disease include cystitis including idiopathic cystitis, urolithiasis, and urethral obstruction.

In certain embodiments, the urinary tract disease is a feline idiopathic cystitis (FIC). FIC is an urinary tract disease in which all diagnostics fail to confirm the cat has another disease. Cats suffering from FIC make frequent attempts to urinate, probably as a result of bladder discomfort, and often are found to have blood in their urine. Signs of urinary tract disease in cats with FIC often resolve within a couple of weeks regardless of treatment, so most veterinarians treat the condition in order to prevent the signs from recurring. In certain embodiments, the urinary tract disease is urolithiasis. Uroliths (stones or calculi found in the urinary tract) have been reported in the urinary system of virtually all animals including dogs, cats, pigs, cattle, rabbits, horses, sheep, goats, deer, whales, birds and many more. The most prevalent types of urinary tract calculi (uroliths) in cats are Calcium Oxalate (CaOx, CaC₂O₄) and struvite (Magnesium Ammonium Phosphate, MAP, MgNH₄PO₄) stones, followed by Urate. The uroliths, which are responsible of a fifth of the total veterinary visits due to urinary tract health in cats, are normally reported in the bladder, although an increased number of renal and ureteral cases have been reported for cats with kidney failure. The incidence of these uroliths is related to the level of saturation of related minerals in the urine. In certain embodiments, the urinary tract disease is urethral obstruction. Urethral obstruction is the most dangerous problem seen in cats with urinary tract disease. Urinary stones are only one of the causes of urethral obstructions. Another common cause is urethral plugs. Urethral plugs consist of a soft, compressible material that contains variable quantities of minerals, cells, and mucus-like protein.

In certain embodiments, the animal is a feline or a canine. In certain embodiments, the animal is a feline. In certain embodiments, the animal is at risk of chronic kidney disease. In certain embodiments, the animal is not known to be at risk of chronic kidney disease. In certain embodiments, the animal suffers from chronic kidney disease. In certain embodiments, the animal is not known to suffer from chronic kidney disease. In certain embodiments, the animal is under a treatment for chronic kidney disease. In certain embodiments, the animal is at risk of urinary tract disease. In certain embodiments, the animal is not known to be at risk of urinary tract disease. In certain embodiments, the animal suffers from urinary tract disease. In certain embodiments, the animal is not known to suffer from urinary tract disease. In certain embodiments, the animal is under a treatment for urinary tract disease. In certain embodiments, the treatment is a dietary therapy.

In certain embodiments, the present disclosure provides for methods of preventing, treating, or delaying the progression of chronic kidney disease (CKD) in an animal. In certain embodiments, the present disclosure provides for methods of preventing, treating, or delaying the progression of urinary tract disease in an animal. In certain embodiments, the methods comprise administering a flavor composition or pet food product disclosed herein (see Sections 2 and 3).

In certain embodiments, the methods further include administering a treatment regimen. In certain embodiments, the treatment regimen is selected from the group consisting of a dietary therapy, hemodialysis, renal replacement therapy, withdrawal of kidney damaging compounds, kidney transplantation, delaying or avoiding kidney damaging procedures, modifying diuretic administration, and combinations thereof. In certain embodiments, the treatment regimen is selected from the group consisting of administering a composition comprising an effective amount of magnesium or a salt thereof, reducing phosphate intake, reducing protein intake, administering polyunsaturated fatty acids, administering a phosphate binder therapy, administering potassium, reducing dietary sodium intake, administering alkali supplements, and combinations thereof. In certain embodiments, the treatment regimen includes any treatment methods described in Jonathan D. Forster, Update on Mineral and Bone Disorders in Chronic Kidney Disease. Vet Clin North Am Small Anim Pract. 2016 November; 46(6):1131-49, the content of which is hereby incorporated by reference in its entirety.

In certain embodiments, the treatment regimen is a dietary therapy. In certain embodiments, the dietary therapy includes a diet selected from the group consisting of a high magnesium diet, a low phosphorous diet, a low protein diet, a low sodium diet, a high potassium diet, a polyunsaturated fatty acids (PUFA) diet, and combinations thereof. In certain embodiments, the dietary therapy is any one of the dietary therapies described in Elliott et al., Dietary therapy for feline chronic kidney disease, Encyclopedia of feline clinical nutrition, 2^{nd} edition, 2015, the content of which is hereby incorporated by reference in its entirety.

In certain embodiments, the present disclosure provides for methods of preventing, treating, or delaying the progression of chronic kidney disease (CKD) and/or urinary tract disease by increasing the water in an animal that receives or has received a urinary diet food that contains sodium chloride or potassium chloride. In certain embodiments, the methods comprise administering a flavor composition or pet food product disclosed herein (see Sections 2 and 3).

In certain embodiments, the flavor composition or pet food product is at an amount of about 10 mg/kcal to about 1000 mg/kcal. For example, and not by way of limitation, the flavor composition or pet food product can be at an amount of about 10 mg/kcal to about 100 mg/kcal, about 20 mg/kcal to about 100 mg/kcal, about 10 mg/kcal to about 200 mg/kcal, about 20 mg/kcal to about 200 mg/kcal, about 50 mg/kcal to about 100 mg/kcal, about 50 mg/kcal to about 200 mg/kcal, about 50 mg/kcal to about 300 mg/kcal, about 100 mg/kcal to about 200 mg/kcal, about 100 mg/kcal to about 300 mg/kcal, about 100 mg/kcal to about 400 mg/kcal, about 100 mg/kcal to about 500 mg/kcal, about 200 mg/kcal to about 500 mg/kcal, about 300 mg/kcal to about 500 mg/kcal, about 200 mg/kcal to about 600 mg/kcal, about 200 mg/kcal to about 700 mg/kcal, about 200 mg/kcal to about 800 mg/kcal, about 300 mg/kcal to about 600 mg/kcal, about 300 mg/kcal to about 700 mg/kcal, about 300 mg/kcal to about 800 mg/kcal, about 400 mg/kcal to about 600 mg/kcal, about 400 mg/kcal to about 700 mg/kcal, about 400 mg/kcal to about 800 mg/kcal or about 500 mg/kcal to about 800 mg/kcal. In certain embodiments, the flavor composition or pet food product is at an amount of about 200 mg/kcal to about 500 mg/kcal, e.g., about 300 mg/kcal. In certain embodiments, the flavor composition or pet food product is at an amount of about 50 mg/kcal to about 200 mg/kcal, e.g., about 100 mg/kcal.

In certain embodiments, the flavor composition or pet food product can be fed to an animal in a constant manner, e.g., where the animal grazes on a constantly available supply of a flavor composition or pet food product. In certain embodiments, the flavor composition or pet food product can be fed to an animal thrice every day, twice every day, once every day, once every two days, once every three days, once every four days, once every five days, once every six days, once a week, once every two weeks, once every three weeks, or once every month. In certain embodiments, the flavor composition or pet food product can be fed to an animal one or more times per day. For example, and not by way of limitation, the flavor composition or pet food can be administered once, twice, three, four, five or more times a day.

### EXAMPLES

The presently disclosed subject matter will be better understood by reference to the following Examples, which are provided as exemplary of the invention, and not by way of limitation.

### Example 1—Determination of the Ability of Flavor Compositions to Increase Water Intake in Healthy Adult Cats

The present example investigates whether the addition of the presently disclosed flavor compositions to the drinking water results in an increase in voluntary free water intake compared to unflavored/blank water in a monadic test situation in healthy adult cats.

### Materials and Methods

Cat drinking test methodology. The monadic test methodology used in the present example is described in Table 1 below.

TABLE 1
Summary of methodology.

| | |
|---|---|
| Test type | Monadic, 1-bottle test. |
| Cats and | Adult cats, n = 24. |
| housing | Cats were housed individually during trial periods. |
| | Cats had free access to water available when not on trial. |
| | Cats were normally divided in two shifts of 12 and take |
| | part in the experiment in a staggered way (i.e. whilst 12 |
| | cats are in Test phase for seven days, the other 12 are |
| | in Rest phase for seven days, and vice versa). |
| Equipment | Cat cup drinkers. |
| Test | Monadic, 1-bottle test. Flavor compositions were offered |
| procedure | in six of the days whereas deionized water was offered |
| | in one of the days, all with two exposures. |
| | Control was made for evaporation loss via installation |
| | of drinkers out of reach from the cats. |
| Test length | 18 hours duration (i.e., 36 hours total exposure |
| | following seven-day crossover). |
| Rest | Following 7 consecutive days of compound exposure/Test |
| | phase, the cats had 7 consecutive days of Rest phase |
| | where they were housed and fed in a social group. |
| Feeding | Cats were offered balanced and nutritionally complete |
| | dry diets as their only caloric source in order to |
| | maximize their free intake of water. |
| | Cats were offered meal portions which were calculated |
| | to meet the individual requirements for each cat (based |
| | on the maintenance of ideal bodyweights and ideal |
| | body condition scores). |
| | Food was offered in two meals: 50% MER (Maintenance |
| | Energy Requirement) offered at the start of the flavor |
| | composition exposure period for 30 min. and the other |
| | 50% MER offered just after finishing the flavor |
| | composition exposure for another 30 min. |

Experimental design. Briefly, six groups of adult cats were split into two shifts of 12 cats which worked alternatively (two days test, two days rest) in a staggered fashion as described in Table 2 below.

TABLE 2
Experimental design used. Product rotation by group.

| Shift 1 | | | Shift 2 | | |
|---|---|---|---|---|---|
| G1 | G2 | G3 | G4 | G5 | G6 |
| A | G | B | D | C | E |
| B | A | C | C | B | D |
| G | F | A | E | D | F |
| C | B | D | B | A | C |
| F | E | G | F | E | G |
| D | C | E | A | G | B |
| E | D | F | G | F | A |

Data collection and analysis. The following measures were collected at specific time intervals throughout each trial: body weights and body condition scores (measured at the beginning of each trial and weekly thereafter); lodge temperatures (one measure taken daily of the temperature in the lodge area); solution temperatures prior to offering; food offered and refused (AM feed and PM feed, daily per cat); solution weights offered and refused (daily per cat); water solution pH; evaporation (two measures taken daily for evaporation of the solutions); and additional comments. A Dunnett's Test was performed at the 500 level using six pairwise comparisons between each of the flavor composition solutions versus deionized water.

Flavor compositions tested. A detailed description of flavor composition solutions tested is presented in Table 3 below. The flavor compositions tested included three flavor compositions based on casein hydrolysate (B, D and F) and three flavor compositions based on amino acid mixtures (C, E and G).

TABLE 3
Flavor compositions tested. ₆₈

| Code | Compound(s) name (s) |
|---|---|
| A | Deionized water |
| C | 50 mM L-Histidine + 50 mM L-Proline + |
| | 5 mM GMP |
| E | 50 mM L-Histidine + 50 mM L-Proline + |
| | 5 mM GMP + 4 ppm Furaneol |
| G | 50 mM L-Histidine + 50 mM L-Proline + |
| | 5 mM GMP + 4 ppm Furaneol + |
| | 10 mM Na₃HP₂O₇ |
| B | 3% Casein + 5 mM GMP |
| D | 3% Casein + 5 mM GMP + 4 ppm Furaneol |
| F | 3% Casein + 5 mM GMP + 4 ppm Furaneol + |
| | 10 mM Na₃HP₂O₇ |

### Results

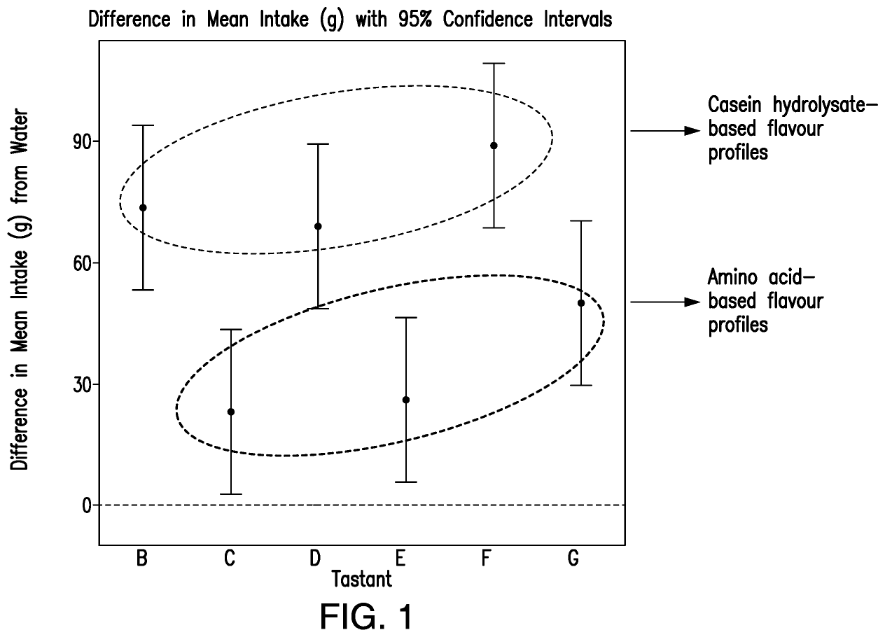

Graphical results obtained for the flavor compositions versus water are shown in FIG. 1. The mean intakes of all six flavor compositions studied were significantly higher than the mean intake of the unflavored/blank water. The flavor compositions based on casein hydrolysate (B=153 g, D=148 g, and F=168 g) had higher intakes compared to the flavor compositions based on amino acid mixtures (C=102 g, E=105 g and G=129 g).

Due to sodium (Na⁺) having a potential impact on the intake of each flavor composition, the levels of sodium in the different flavor compositions were calculated. Sodium in the flavor composition studies was derived from the casein hydrolysate, the GMP (disodium form), or from the pyrophosphate (trisodium pyrophosphate). The casein hydrolysate used in this study contained a level of sodium of 2.5%. The Na⁺ level in the other two components could be calculated by stoichiometry. Therefore, the flavor compositions based on casein hydrolysate (B, D and F) had a sodium content of 0.98 g/L, 0.98 g/L and 1.67 g/L; respectively, whereas the sodium content of the amino acid-based flavor compositions (C, E and G) was 0.23 g/L, 0.23 g/L and 0.92 g/L; respectively.

All of the flavor composition solutions had a significantly higher intake than deionized water, which meant that in a monadic situation, the inclusion of a flavor composition in the water encouraged the cats to drink more than it would do with unflavored/blank water. The extent of this increase in total intake per cat per day depended on the flavor composition type, with the flavor compositions containing casein hydrolysate having higher intakes than the amino acid-based flavor compositions.

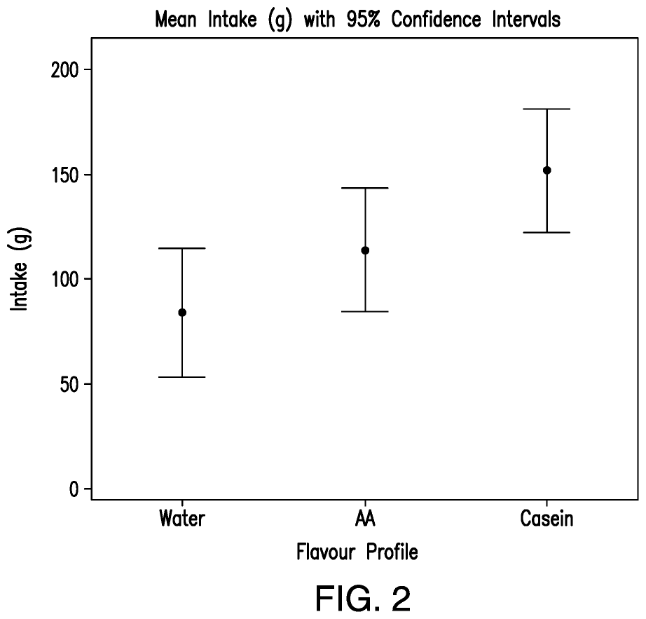

A complementary analysis of the influence of flavor composition type (casein hydrolysate-based or amino acid-based) was performed. The representation of mean intake by flavor composition type was not done in the Dunnett's test, but with Tukey's. Significant differences between profile types were found, p<0.001. FIG. 2 illustrates the means with 95% confidence intervals. All differences were significant.

### Conclusion

It was possible to increase the water intake of cats in a monadic situation. All six flavor compositions had a significantly higher intake than water when comparing their mean intakes using Dunnett's test. These results demonstrated that the inclusion of flavor compositions in water can increase water consumption in healthy adult cats.

### Example 2—Determination of the Ability of Flavor Compositions to Increase Water Intake in Cats with Early Stage Chronic Kidney Disease (CKD)

The present example investigates whether the addition of the presently disclosed flavor compositions to the drinking water results in an increase in voluntary water intake in cats identified with asymptomatic and stable early CKD (IRIS stages 1 or 2; Renal cats). Currently, it is not known if it is possible to increase water intake via addition of the presently disclosed flavor compositions to the water in cats with early stages of CKD, as their drinking behavior is likely to be different due to physiological and/or clinical reasons.

### Materials and Methods

Cat drinking test methodology. This methodology involved two 22-hour exposures for water and each flavor solution with 16 cats with stable early CKD and is described in Table 4.

TABLE 4
Summary of the drinking test methodology.

| | |
|---|---|
| Test type | Monadic, 1-bottle test. |
| Cats and | Cohort of cats with stable early CKD, n = 16. |
| housing | Cats were housed individually during trial periods |
| | (24 hour each of the two days, with breaks during |
| | the day summing up to 2 hours - total 22-hour exposure). |
| | Cats did not have any access to water during |
| | these 2-hour breaks. |
| | Cats were divided into six groups of 2 or 3 individuals |
| | (due to individual housing availability) and took part |
| | in the experiment in a staggered way (i.e. whilst one |
| | group of cats were in Test phase for two days, all the |
| | other cats were in Rest phase). |
| Equipment | Cat cup drinkers. |
| | Ceramic bowls were used for cats not adapting to the |
| | cat cup drinkers. |
| Test | Monadic, 1-bottle test. Three flavor compositions |
| procedure | were offered on three tests whereas plain mineral |
| | water (i.e. Highland Spring), was offered in another |
| | two tests (pre-feed and baseline). All tests included |
| | two exposures. An additional baseline plain water |
| | intake phase at the end of the trial was also added |
| | (see below for justification). |
| | 350 mL of solution (either plain or flavored) was |
| | offered to every cat on each Test day. |
| | Control was made for evaporation loss via |
| | installation of drinkers out of reach of the cats. |
| Test length | Two 22-hour durations (i.e. 44 hours total exposure). |
| Rest | Following 2 days of compound exposure/Test phase, |
| | cats had 9 consecutive days of rest where they were |
| | housed and fed in a social group. |
| Feeding | Cats were offered wet and dry diets as their only |
| | calorie source. Some cats had water added to their |
| | diet on Rest days. |
| | On Test days, cats were offered their food as dry |
| | format to enhance their water intake (except some |
| | cats where different conditions were allowed). |
| | Cats were offered meal portions which were calculated |
| | to meet the individual requirements for each cat |
| | (maintenance of ideal bodyweight and ideal body |
| | condition score - % MER). |
| | Food was offered in two meals for most cats, |
| | but 2 cats had three meals. |

Early CKD cats cohort—health, behavioral and water testing background. A total of 16 cats (9.4 years median age, range 6.5-13.3 years at the start of the trial) took part in this study. Cats had been diagnosed with early-stage CKD, which was clinically stable at IRIS stage 1 (11/16 cats) and IRIS stage 2 (5/16 cats) based on serum creatinine, symmetric dimethylarginine (SDMA), systolic blood pressure, proteinuria, kidney ultrasound, and urinary tract imaging. Among the IRIS 2 cats, three of them had renoliths/ureteroliths and one had a bladder stone. Four of the 16 cats had a clinical history of feline idiopathic cystitis. The cohort of cats was habituated to the equipment used in the study in advance.

### Experimental Design

Initially, a trial with a “pre-feed” water phase (Phase 1; Table 5) was performed for the cats to get used to the cat cup drinkers. The pre-feed phase aimed to only collect behavioral data and habituate the cats to the drinkers and lodging, but not to use this data to measure baseline water intakes. The second phase of the trial was the water baseline test, conducted without the addition of the presently disclosed flavor compositions. After this phase, the next three tests (Phases 3, 4, and 5) offered the three different flavor compositions tested in a randomized way (see Table 5), giving a total of five test blocks for each cat:
Phase 1: Water T0 (habituation & training & behavioral observations—data not to be used in the analysis).
Phase 2: Water T0 (baseline water—benchmark to compare with the 3 flavor compositions tested).
Phase 3: Flavor Composition option (X, Y or Z, depending on randomized groups).
Phase 4: Flavor Composition option (X, Y or Z, depending on randomized groups).
Phase 5: Flavor Composition option (X, Y or Z, depending on randomized groups).

For each phase, the 16 cats were tested in a staggered way (Table 5) due to the availability of lodges needed for the individual drinking tests. For each 48-hour test period, each cat was individually lodged for a total of 44 hours. (Two-hour socialization periods without access to any water was provided for each 24-hour period during the test phase. Each of the drinking tests consisted of a repeat exposure where the cats had the water solutions offered to them monadic ally. Each Test phase was followed by a 9-day Rest phase. On Rest days, the cats lived normally in the larger social group with free access to water.

TABLE 5
Solution rotation by cat group.

| | | Phase 1 | Phase 2 | Phase 3 | Phase 4 | Phase 5 |
|---|---|---|---|---|---|---|
| Group 1 | A-B-C-D | Water | Water | Flavor 1 | Flavor 2 | Flavor 3 |
| Group 2 | A-C-D-B | Water | Water | Flavor 2 | Flavor 3 | Flavor 1 |
| Group 3 | A-D-B-C | Water | Water | Flavor 3 | Flavor 1 | Flavor 2 |
| Group 4 | A-D-C-B | Water | Water | Flavor 3 | Flavor 2 | Flavor 1 |
| Group 5 | A-B-D-C | Water | Water | Flavor 1 | Flavor 3 | Flavor 2 |
| Group 6 | A-C-B-D | Water | Water | Flavor 2 | Flavor 1 | Flavor 3 |

Diets and feeding regimen. Each cat in this cohort had different food formats (dry or wet) depending on its medical and/or behavioral needs. The water content of the diets was 78.3% for the wet diet and 4.7% for the dry diet, which were used for total water intake calculations. Most of the cats in this cohort were fed in a dry-wet mixed feeding regime, with each format providing approximately 50% of their maintenance energy requirement. Exceptions to this were four cats with previous history of feline idiopathic cystitis which received all their meals as wet as advised by the veterinarian. Some of these cats also had extra water added to their meals. Two cats had three meals a day instead of two. This feeding regime was kept for rest days only, however, and the cats were offered dry food whilst in the lodges on Test days.

Water solutions tested. A description of flavor composition solutions tested is presented in Table 6 below. Further details regarding the flavor mixes:
X: composed of a cat umami-active amino acid (L-histidine) at 100 mM and a cat non-umami active amino acid (L-proline) at 100 mM.
Y: composed of a cat umami-active amino acid (Glycine) at 266 mM (or 2%) and a cat non-umami active amino acid (L-threonine) at 20 mM.
Z: composed of a cat umami-active amino acid (Glycine) at 266 mM (or 2%), a cat non-umami active amino acid (L-threonine) at 20 mM and furaneol at 4 ppm.
   The water used in the drinking tests was mineral spring water (Highland Spring 1.5 L) to avoid taste variability. The flavor compositions tested were prepared with the same water and were selected based on specific requirements of cats with IRIS stages 1 or 2 CKD. These restrictions included no addition of phosphorous or calcium that could disrupt the Ca:P ratio, considerations on amino acid type, and limitations of sodium and other salt content.

TABLE 6
Solutions tested.

| No. | Code | Solution | Letter | Compound (s) name (s) |
|---|---|---|---|---|
| 1 | T0 | Baseline | A | Mineral water Highland Spring |
| 2 | X | Flavor 1 | B | 100 mM L-Histidine + |
| | | | | 100 mM L-Proline |
| 3 | Y | Flavor 2 | C | 2% Glycine + 20 mM L-Threonine |
| 4 | Z | Flavor 3 | D | 2% Glycine + 20 mM L-Threonine + |
| | | | | 4 ppm Furaneol |

Data collection and analysis. The following measures were collected at specific time intervals for each cat throughout the study. Body weights and body condition scores were measured at the beginning of each phase and weekly thereafter. The amount of food offered and refused at both AM and PM feeds and any extra meals, daily. The amount of water and flavor composition solutions offered and refused on each test day. The flavor composition solution pH, evaporation, and additional comments (e.g., any observed behavioral traits, or medication administered to individual cats on trial) were also noted. All raw water and flavor solution intake data were corrected for evaporation and any reported spillages.

Two variables were used for the analysis: (1) free water solution intake and (2) total water intake, which is calculated by the sum of free water intake and the water present in the diet ingested. Due to the influence of body weight and body surface area on water intake, two calculated values of grams per Kg of body weight and grams per Kg of metabolic body weight (kg BW^{0.711}) were included in the data set. A linear mixed effects model was fit using the data collected during the baseline water and the water with the added flavor compositions. The variables group, flavor compositions and the interaction between the two were the fixed effects and the individual animal was the random effect. A likelihood ratio test was also conducted to assess the significance of group and the interaction to the model. If these were deemed to not be significant, only the flavor composition variable was used as the fixed effect. If group was not deemed to be significant, then no period effect was found. Residuals were visually assessed for the assumption of normality. If they were deemed to not be normally distributed, a log transformation was applied or an appropriate non-parametric test. If the variances were not deemed to be homogeneous, a weighting was applied to the flavor composition. Estimated means were extracted and reported with 95% CI. A Dunnett's test was then conducted comparing the flavor composition intakes back to the baseline water. Estimated differences or fold changes were obtained and reported along with 95% CI and p-values. A statistically significant difference was determined by a p-value ≤0.05. All analyses were performed using R version 3.6.3.

### Results

Four cats fed 100% wet diet had very low water intakes during the pre-feed phase (Phase 1). Following consultation with the attending veterinarian, these four cats transitioned to mixed dry-wet regimen during the following phases, so all cats were in a wet-dry regime during the remainder of the study. Also, as the study progressed and the free water and total water intakes were examined after each of the phases, it was observed that some of the cats increased their water intake as the trial progressed while others did the opposite. It was flagged that it might be difficult to separate the flavor composition effect from the behavioral effect of lodging. For this reason, it was decided to run an additional water baseline phase (Phase 6) at the end of the study to be able to detect a difference in drinking behavior from the beginning to the end of the study (see Table 7 below).

TABLE 7
Final six-phase experimental design.

| | | Phase 1 | Phase 2 | Phase 3 | Phase 4 | Phase 5 | Phase 6 |
|---|---|---|---|---|---|---|---|
| Group 1 | A-B-C-D | Water | Water | Flavor 1 | Flavor 2 | Flavor 3 | Water |
| Group 2 | A-C-D-B | Water | Water | Flavor 2 | Flavor 3 | Flavor 1 | Water |
| Group 3 | A-D-B-C | Water | Water | Flavor 3 | Flavor 1 | Flavor 2 | Water |
| Group 4 | A-D-C-B | Water | Water | Flavor 3 | Flavor 2 | Flavor 1 | Water |
| Group 5 | A-B-D-C | Water | Water | Flavor 1 | Flavor 3 | Flavor 2 | Water |
| Group 6 | A-C-B-D | Water | Water | Flavor 2 | Flavor 1 | Flavor 3 | Water |

From the 16 cats that started the study, three were removed from the study at different points. Thus, the two water baselines (Phase 1—water baseline 1 and Phase 6—water baseline 2) plus the three flavor composition phases (Phases 3, 4 and 5) were completed by 12 cats. The statistical analysis of the intake of the flavor compositions included 13 cats, as one cat had been exposed to the first water baseline and all the flavor compositions, but did not participate in the second water baseline.

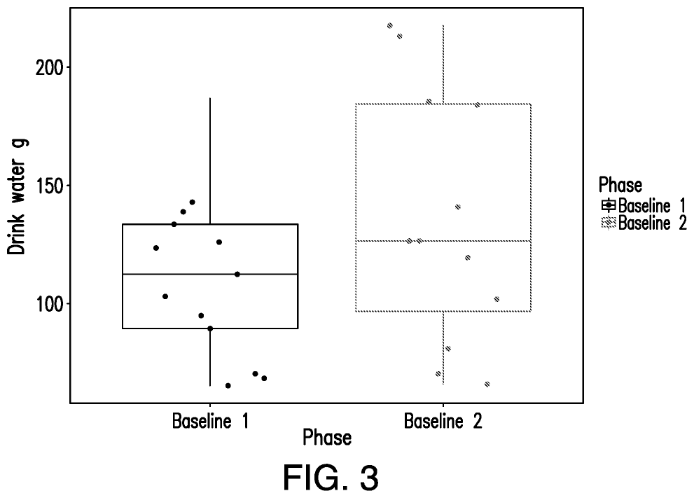

The comparison between the free water intake before the study (Phase 2—Baseline 1) and at the end of the study (Phase 6—Baseline 2; Table 8, FIG. 3) revealed a difference between intakes of 21.9 g, which did not reach significance (p-value was 0.074). So, evidence of a change in response during the study was not found. Based on the above results, Baseline 1 was therefore taken as a valid benchmark to assess the effect of flavor composition solutions on water intake and it was used in all subsequent analyses.

When analyzing the free water intake for water Baseline 1 and the flavor composition solutions, a prior statistical check (likelihood ratio test) was conducted to determine if there was any cat group effect or time of testing effect. The result was not significant (p-value 0.460). A second statistical check was conducted to determine if there was any flavor composition group interaction effect, and again this was not significant (p-value 0.497).

TABLE 8
Water intake for the different Flavor Compositions showing mean and 95% confidence intervals (CI) and p-values
for the differences in water intake relative to plain water intake at the start of the study (baseline 1).

| | Water | Water | | | | | | | |
|---|---|---|---|---|---|---|---|---|---|
| | (start) | (End) | | Y | | Z | | X | |
| | Mean (CI) | Mean (CI) | P | Mean (CI) | P | Mean (CI) | P | Mean (CI) | P |
| Free water | 112 | 134 | 0.074 | 124 | 0.302 | 129 | 0.051 | 137 | 0.028 |
| intake | (84.6, 139) | (106, 162) | | (94.8, 154) | | (100, 157) | | (105, 170) | |
| (g/d) | | | | | | | | | |
| Total | 118 | 139 | 0.082 | 130 | 0.303 | 135 | 0.052 | 143 | 0.027 |
| water¹ | (91.9, 144) | (112, 166) | | (102, 158) | | (108, 161) | | (112, 174) | |
| intake | | | | | | | | | |
| (g/d) | | | | | | | | | |
| Total | 26.3 | 30.2 | 0.099 | 29.1 | 0.347 | 29.7 | 0.054 | 31.5 | 0.044 |
| water | (20.0, 32.3) | (23.8, 36.7) | | (21.7, 36.6) | | (22.8, 36.6) | | (23.7, 39.3) | |
| intake | | | | | | | | | |
| (g/kg | | | | | | | | | |
| BW × d) | | | | | | | | | |
| Total | 9.2 | 10.4 | 0.119 | 10.2 | 0.372 | 10.3 | 0.304 | 10.9 | 0.050 |
| water | (6.5, 11.9) | (7.7, 13.1) | | (6.74, 13.7) | | (6.83, 13.8) | | (7.46, 14.4) | |
| intake | | | | | | | | | |
| (g/kg | | | | | | | | | |
| BW^{0.711} × d) | | | | | | | | | |

¹Total water intakes include water intake derived from the meal.

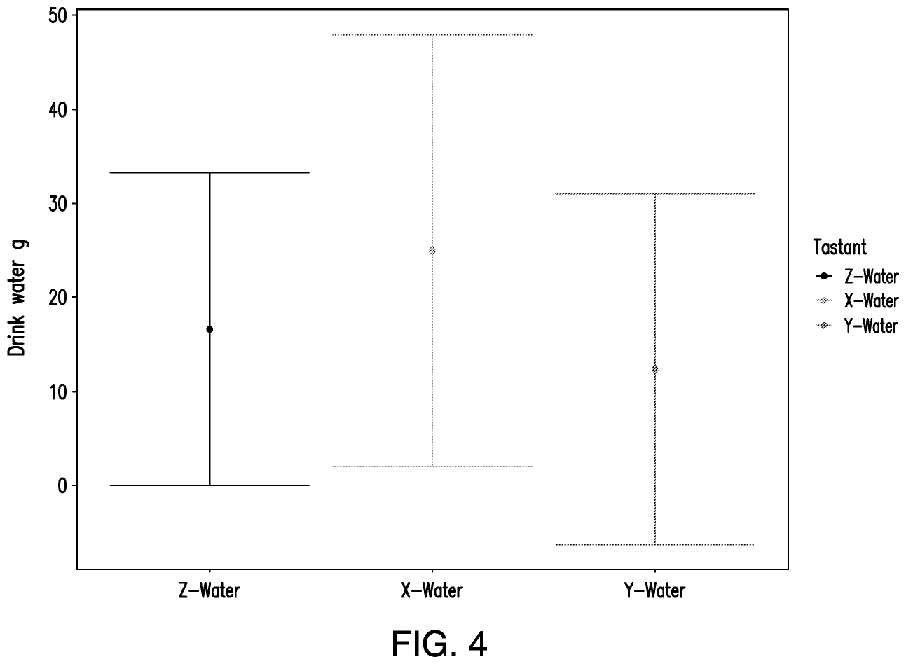

Table 8 and FIG. 4 provide mean free water intakes for the different flavor compositions offered. When analyzing each of the flavor compositions versus the water baseline at the start of the study using Dunnett's test, a significant difference was only observed for one flavor composition, X (difference of 25 g versus water, p-value 0.028), but not for the other two (difference of 12.4 g versus water, p-value 0.302 for Y; difference of 16.6 g versus water, p-value 0.051 for Z). The difference for Z indicated a trend. The percentage increase of free water intake versus baseline achieved by each of the flavor compositions was 10.7% for Y, 15.2% for Z and 22.3% for X.

For the total water intake (free water intake+water intake from food), the group and flavor composition*group interaction effect were checked, and they were not significant. The differences between the flavor compositions and water were significant for X (difference of 25 g versus water, p-value 0.027), but not significant for the other two (difference of 12.4 g versus water, p-value 0.303 for Y; difference of 16.6 g versus water, p-value 0.052 for Z), again indicating a trend for Z. The percentage increase of total water intake versus baseline achieved by each of the flavor compositions was 10.2% for Y, 14.4% for Z and 21.2% for X. The results when intakes were expressed as grams water intake per Kg of body weight revealed differences between the flavor compositions and water of 2.84 g/Kg BW (p-value 0.347) for Y; 3.42 g/Kg BW (p-value 0.054) for Z; and 5.24 g/Kg BW (p-value 0.044) for X. Again, X resulted in significantly increased intake per kg BW.

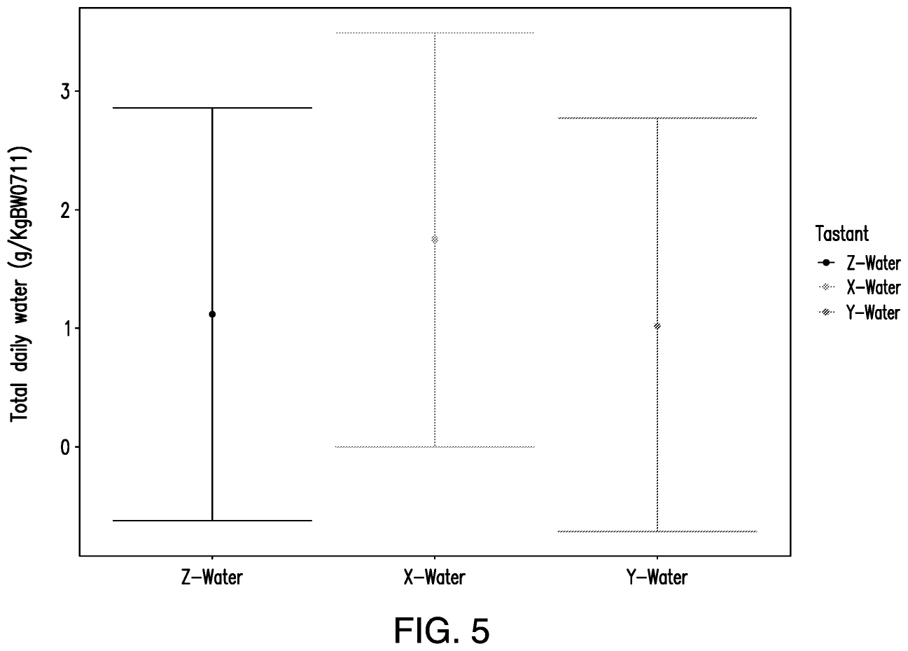

The results expressed as grams intake per Kg of metabolic body weight (g*Kg BW^{0.711}) also identified X as the flavor composition that significantly increased total water intake (p=0.050; see Table 8 and FIG. 5). The differences between the flavor compositions and water were 1.03 (p-value 0.372) for Y; 1.12 (p-value 0.304) for Z; and 1.75 g/Kg metabolic BW (p-value 0.050) for X.

### Discussion

Among the three flavor compositions tested, X composed of a cat umami-active amino acid (L-histidine) and a cat non-umami active amino acid (L-proline) both at high concentration (100 mM), significantly increased the water intake of the cohort of cats with early stages of CKD for all the calculated parameters.

### Conclusions

Flavor composition X was the best-performing of the 3 flavor compositions tested in this study, significantly increasing all measures of water intake in a cohort of 16 cats with early and stable CKD (IRIS 1-2) during a 48-h monadic exposure. These results suggest that the inclusion of X in water can influence water intake in cats with early stages of CKD.

### Example 3—Effect of Offering a Functional Drink in Addition to Water to Promote Additional Water Intake and Diuresis in Cats Fed Dry Diets

The present example investigates the effect of offering water enhanced with certain flavor compositions disclosed herein in addition to plain water to cats fed dry diets on total water intake and urine parameters.

To promote water intake and urine dilution in cats fed dry diets and in order to decrease calcium oxalate relative supersaturation (RSS CaOx) and MAP, the strategy commonly used (at least in urinary diets) is to increase water intake by feeding wet diets or by increasing dietary sodium chloride or potassium chloride in dry diets. However, alternative strategies to promote even greater water intake and urine dilution are interesting to explore.

Thus, there was a need for developing a flavor composition that could increase spontaneous water in cats, promote urine dilution, and lower relative supersaturation (RSS

In addition to the effects on water intake and urine parameters, the present example also assessed whether the effects were similar for two diets promoting different levels of spontaneous water intake. In particular, it was assessed whether i) offering a palatable drink on top of a maintenance (regular) dry diet could be a substitute to offering a dry diet intended for urinary tract disease to increase water intake and diuresis, and decrease the risk of urine crystal formation, and ii) offering a palatable drink on top of a dry diet intended for urinary tract disease had additional benefits compared to the dry diet intended for urinary tract disease alone. It was hypothesized that, when this palatable drink was offered ad libitum, cats would consume a higher daily volume of liquid, leading to higher urine volumes and lower RSS values, when fed both the regular or the diet intended for urinary tract disease.

### Materials & Methods

Two commercially available dry feline diets were used for this protocol: a dry diet intended for urinary tract disease and a regular dry cat food diet The two diets were fed sequentially to 14 healthy adult cats, divided into 2 panels, which completed standard digestibility/RSS trials in an incomplete crossover design.

In addition to the classical measurements for digestibility and RSS, the intakes of water supplemented with flavor composition F and unsupplemented were recorded (weighed) individually. The weights (grams) of liquids measured were converted into volumes (mL) using the density of the given liquid (which was measured for flavor composition F).

The solution was prepared the same day or the day before the test for freshness purposes. When it was prepared the day before, it was kept in the refrigerator at 4° C. until the feeding test.

The preparation of flavor composition F was more difficult than expected due to the slow dissolution of the components during stirring. In addition, some precipitate could be seen in the bowl of cats after several hours after introduction to the cats, leading the animal caretakers to regularly stir the solution to ensure proper dissolution during the feeding trial.

### Statistical Analyses

A linear mixed model (mixed procedure of SAS) was used to assess the influence of diet type, Supplemented drink (e.g., flavor composition F) availability (e.g., SUPP, NO SUPP) and cat panel and their respective interactions as fixed effects on several parameters (e.g., food and liquid intakes, and urinary parameters). The cat was included as a random term, as each cat was its own control. According to the residual distribution of each model, output variables were ranked or not. The difference between 2 levels of fixed effects was assessed by the Scheffe test (e.g., adjustment for multiple comparisons). To assess the difference in flavor composition F intake for each diet, the model only included the diet as a fixed effect and the cat as a random term. Data are expressed as Least Square Means±SE (except if indicated otherwise). Significance level was set at 0.05.

### Results—Discussion

Following consultation 4 cats (one from the diet intended for urinary tract disease and three from the regular diet) were excluded because meeting the exclusion rule for the RSS calculation (e.g., only one urine sample was collected over the collection period). Therefore, the final statistical model included 13 cats for urine related parameters with the diet intended for urinary tract disease, and 11 cats for urine-related parameters with the regular diet. For diet and liquid intake data, all cats (n=14) were included.

The results and corresponding statistical analyses are reported in Tables 9 and 10, respectively, which are provided below. There was no panel effect for any of the parameters.

TABLE 9
LS Means, SE or Medians obtained for the cat panels for each diet with or without F availability.
Dry diet intended for urinary tract disease is labeled as “UrinaryDiet.”

| | | | | | Urinary | |
|---|---|---|---|---|---|---|
| | | Regular | | Urinary | Diet | |
| LS Means | Unit | NoSupp | RegularSupp | DietNoSupp | Supp | SE |
| BW | kg | 5.52 | 5.52 | 5.44 | 5.46 | 0.37 |
| kcal_IntakeBW\hat{ }0.71 | kcal | 65.5 | 56.0 | 76.8 | 68.4 | 4.2 |
| Na_TotInt | g/d | 0.25 | 0.45 | 0.49 | 0.64 | 0.03 |
| H20_Intake (H2O Drink) | mL/kg/d | 22.4 | 5.21 | 27.5 | 9.04 | 1.6 |
| Flavor composition F_Int | mL/kg/d | | 28.4 | | 26.2 | |
| H20 + Flavor composition F Intake | mL/kg/d | 22.4 | 33.6 | 27.5 | 35.2 | 2.5 |
| (H2O Drink + Flavor composition F) | | | | | | |
| TotLiquid_Int (H2O Drink + H2O | mL/kg/d | | 0.50 | | 0.28 | |
| Drink + H2O Feed + Flavor | | | | | | |
| composition F) | | | | | | |
| USG | | 1.064 | 1.049 | 1.058 | 1.053 | 0.004 |
| Ur_vol (median) | mL/kg/d | 9.3 | 13.3 | 12.3 | 15.2 | — |
| U_pH | | 6.50 | 6.77 | 6.28 | 6.45 | 0.06 |
| RSS_CaOx | | 3.39 | 1.73 | 3.15 | 2.11 | 0.35 |
| RSS_MAP (median) | | 1.78 | 0.79 | 0.30 | 0.43 | — |

TABLE 10
P values obtained from the general linear model.

| | | effect of flavor | |
|---|---|---|---|
| | Effect of diet | composition F | diet*(flavor |
| | Urinary/ | with/ | composition F) |
| | Regular | without | interaction |
| BW kg | 0.0001 | 0.5346 | 0.5069 |
| kcal from diet/BW\hat{ }0.711 | <.0001 | 0.0007 | 0.8108 |
| H2O volume intake/kg/d | 0.0011 | <.0001 | 0.6208 |
| Flavor composition F | 0.3145 | | |
| vol intake/kg/d | | | |
| H2O + Flavor | 0.0639 | <.0001 | 0.3361 |
| composition F volume | | | |
| intake/kg/d | | | |
| Urine volume/kg/d | 0.0005 | 0.0022 | 0.0444 |
| USG | 0.7649 | 0.0015 | 0.0936 |
| UpH | <.0001 | <.0001 | 0.2467 |
| RSS CaOx | 0.7524 | <.0001 | 0.1683 |
| RSS MAP | <.0001 | 0.2564 | 0.0157 |

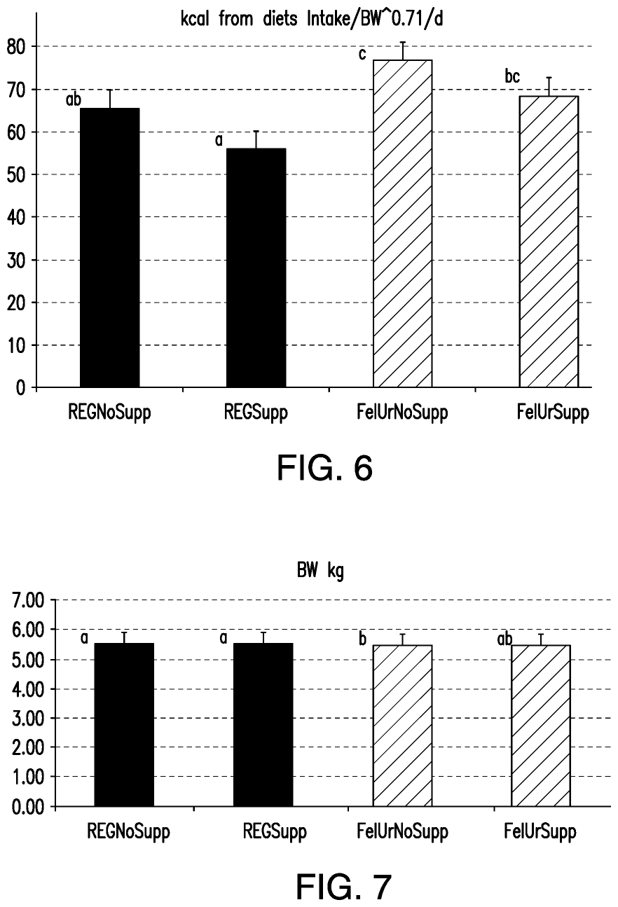

It was observed that the caloric intake from the diet itself, was overall different according to the Diet (Urinary>Regular) and significantly lower when flavor composition F was available (FIG. 6). Further, a difference in body weight (BW) between diets was found. Notably, said difference was not according to flavor composition F supplementation. However, BW variation across the entire duration of the study (8 weeks) ranged from −2.7% to +4.1% in the overall population and for a given RSS test (2 weeks), it ranged between −6.1% and +4.7%. Therefore the difference in BW between diets was not considered biologically relevant (FIG. 7).

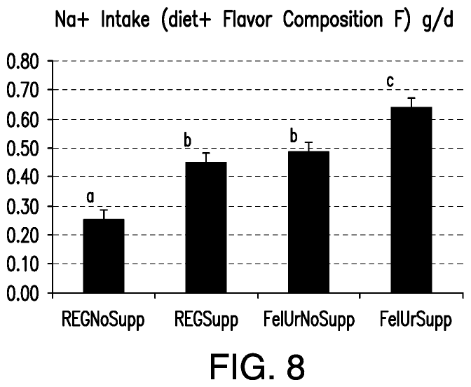

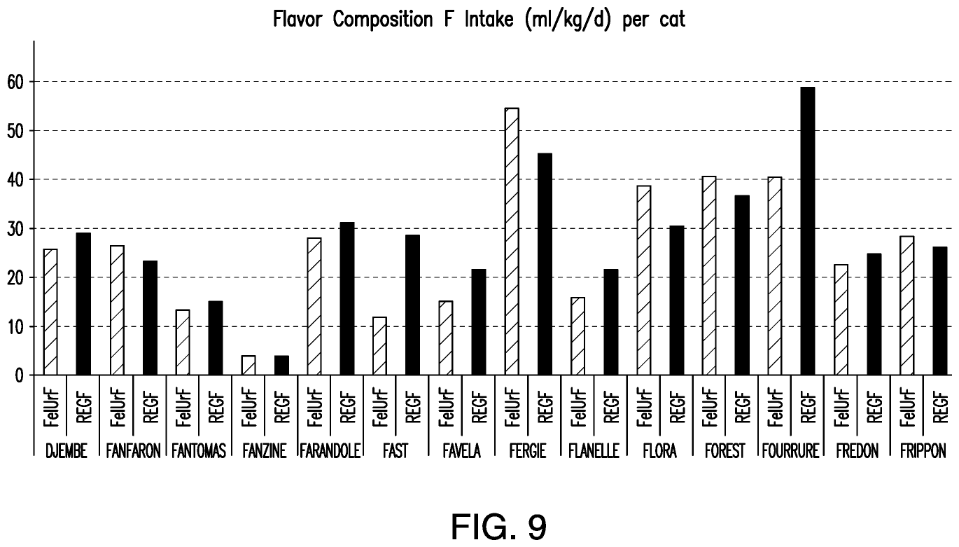

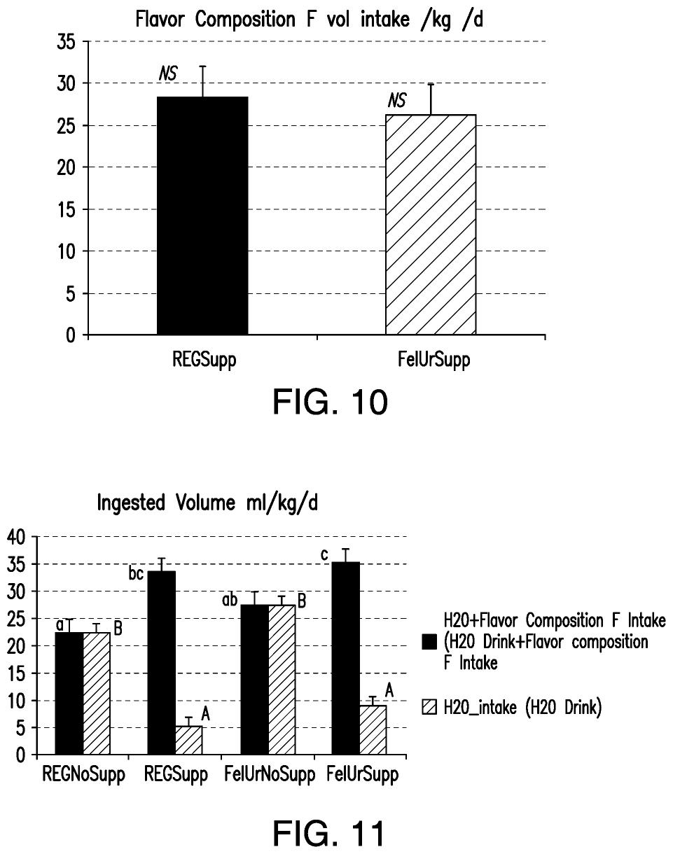

Next, it was observed that, for both diets, the sodium intake (g/day) was significantly higher when flavor composition F was offered (FIG. 8). Intake of flavor composition F was variable among the cats for a given diet, ranging from 4 ml/kg/d to 58.8 ml/kg/d (FIG. 9). Overall flavor composition F intake (mL/kg/d) did not differ according to the diet tested (Urinary or Regular) (p=0.32) (FIG. 10). Notably, when flavor composition F was offered in addition to water, spontaneous water intake (mL/kg/d) was significantly lower. When overall liquid intake (water+flavor composition F) was considered, it was significantly higher for both diets when flavor composition F was offered (FIG. 11).

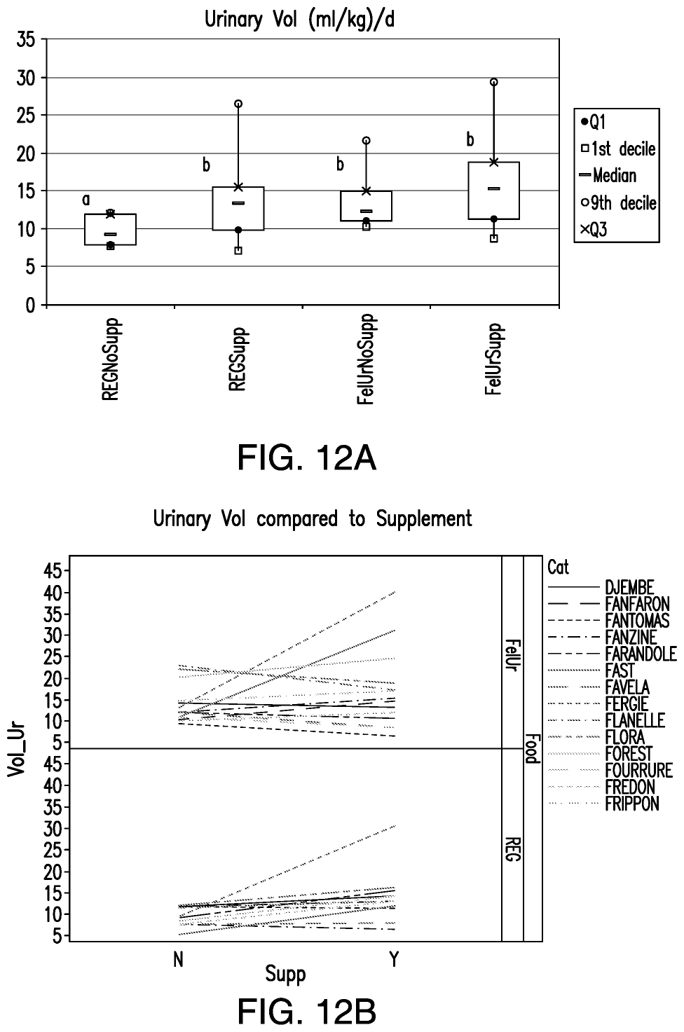

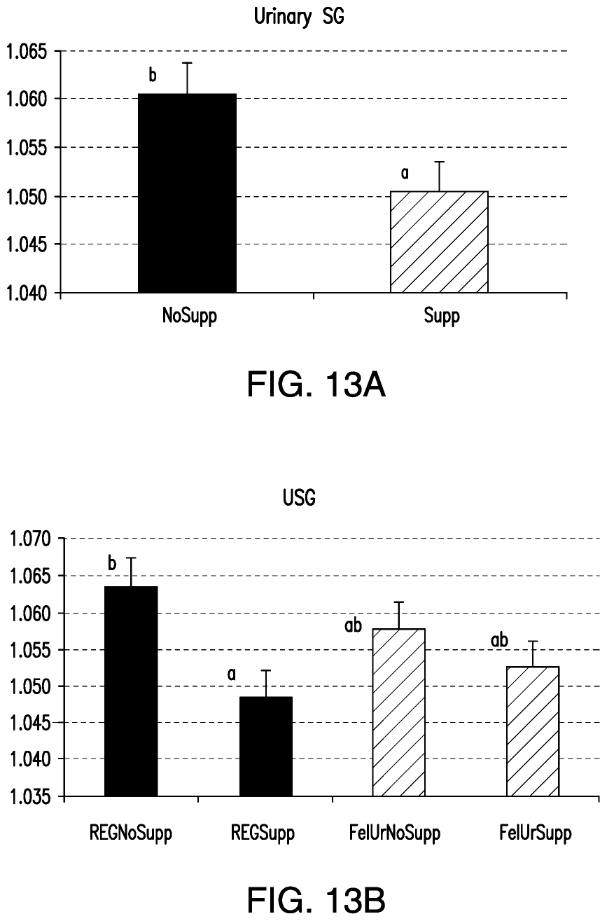

The urinary parameters were also analyzed. Urinary volume (mL/kg/d) increased significantly when flavor composition F was offered for the Regular diet. This was not found for the Urinary Diet despite a higher liquid intake (FIGS. 12A and 12B). Further, there was a significant decrease of the Urinary Specific Gravity when the supplement drink flavor composition F was offered (FIGS. 13A and 13B).

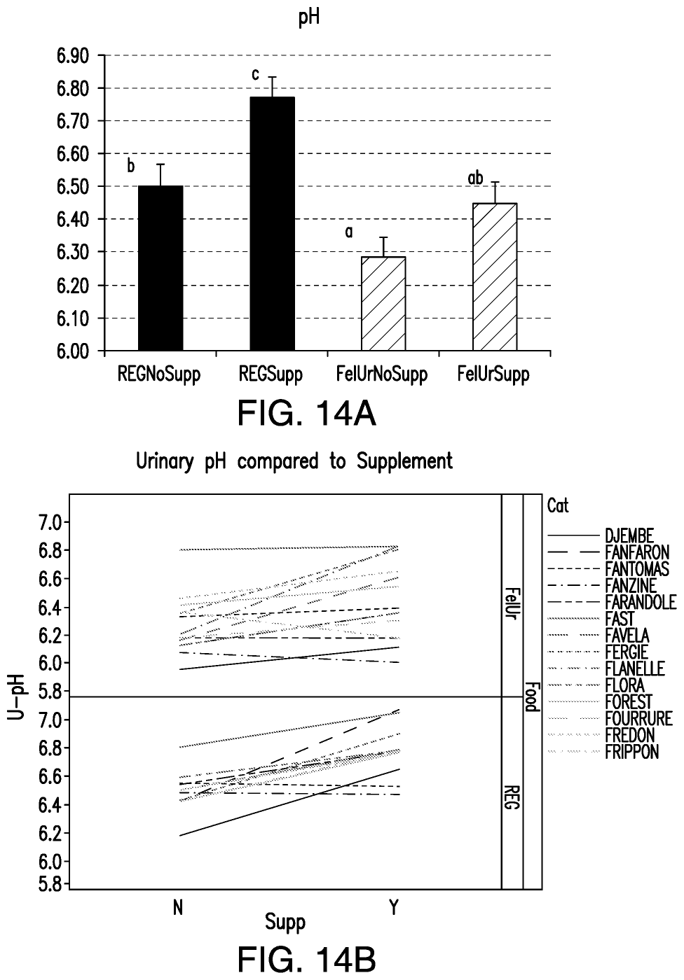

It was observed that Urine pH was significantly higher with the Regular diet than with the Urinary diet. When the flavor composition F was offered urine pH increased significantly for the Regular diet (FIGS. 14A and 14B).

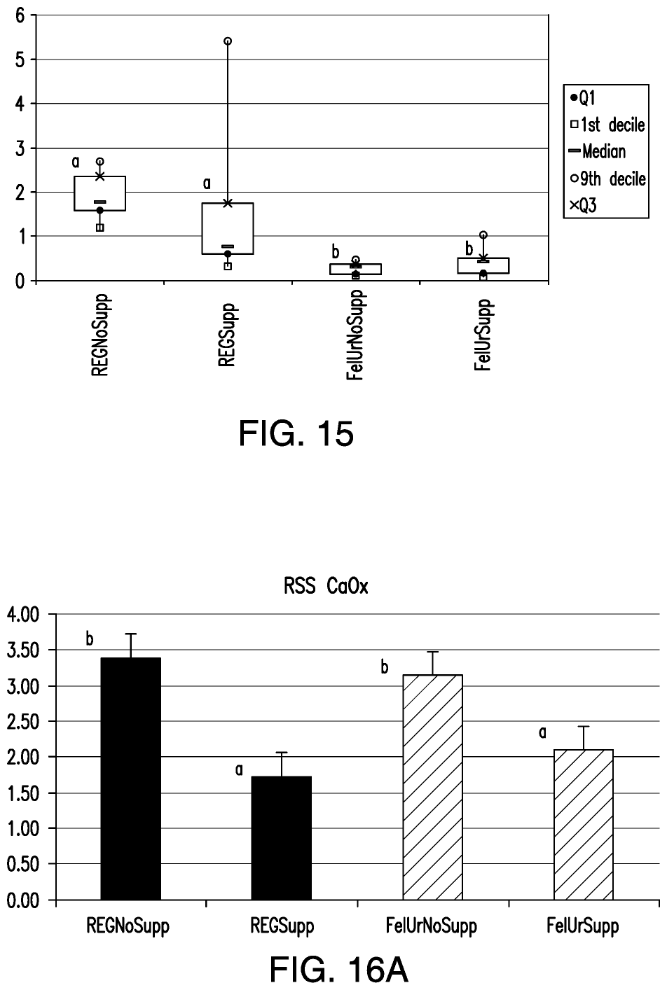

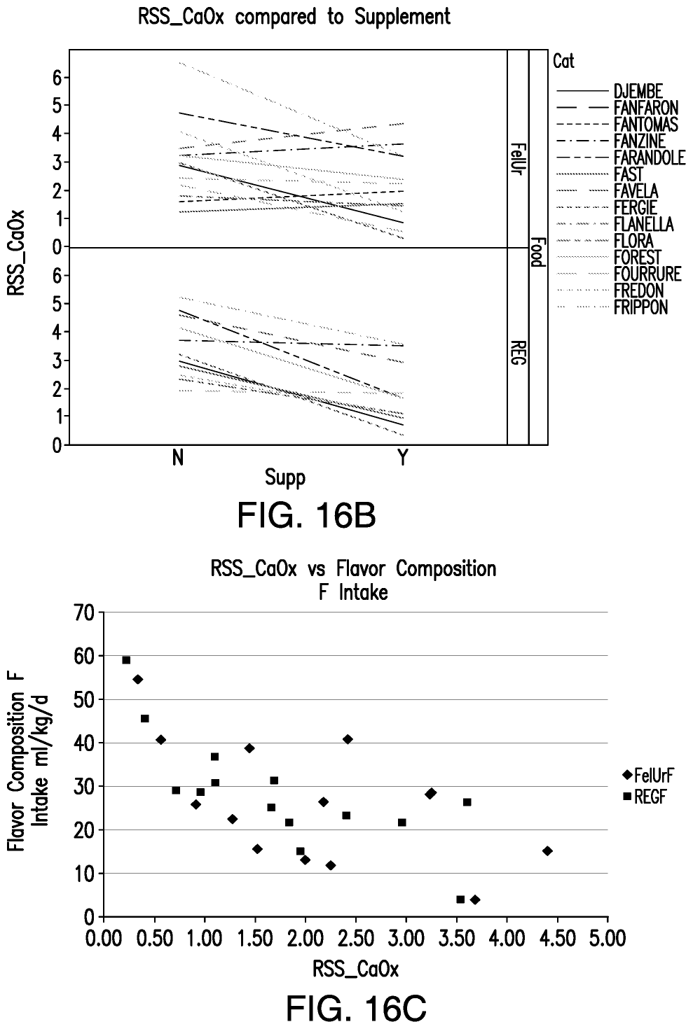

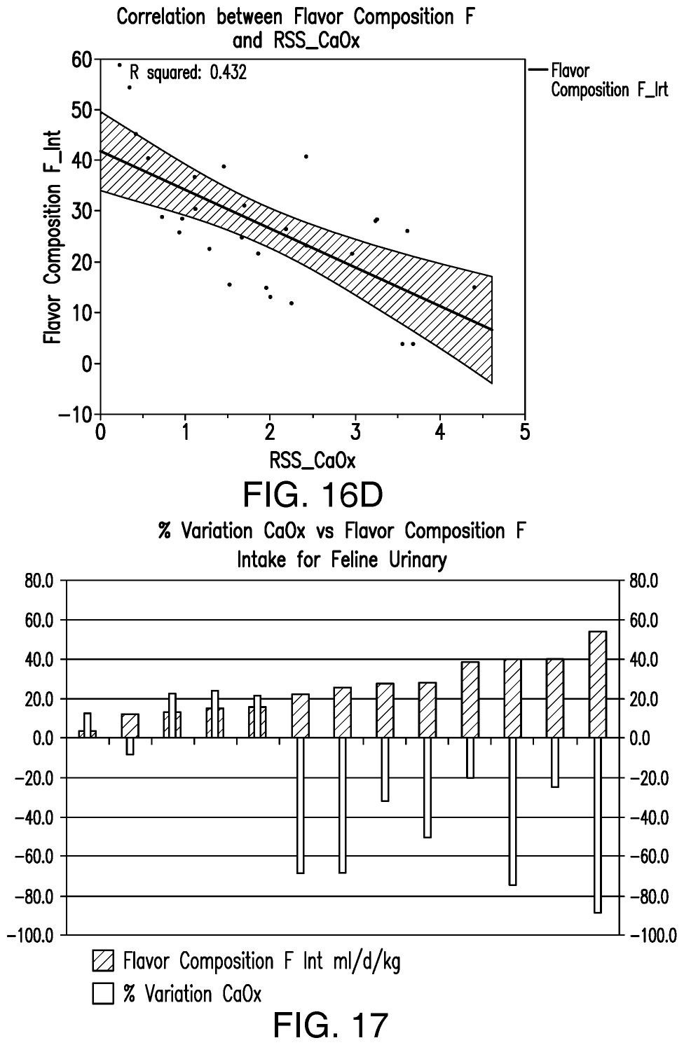

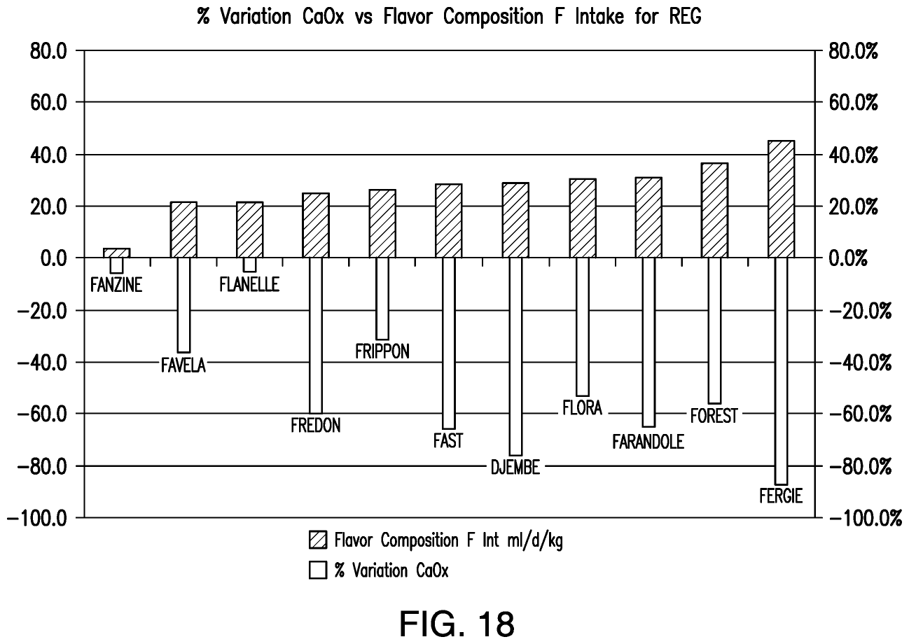

RSS analysis were also conducted. RSS MAP was significantly higher with the Regular diet than with the Urinary diet and was unaffected by the supplement (FIG. 15). Further, RSS CaOx for both diets were significantly lower when flavor composition F was offered (FIG. 16A). For most cats, RSS CaOx decreased when the supplement was offered (FIG. 16B). Although not statistically analyzed, all cats drinking ≥20 mL/kg/day of flavor composition F had a lower RSS CaOx regardless of the diet. For the Regular diet, 4/5 cats drinking <20 mL/kg/day of flavor composition F had an increase in RSS CaOx, and 1/5 cat had a decrease in RSS. Notably, there was a significant inverse linear correlation (R2=0.43) between the intake of flavor composition F and the RSS value of the cat (FIGS. 16C, 16D, 17, and 18).

### Conclusions—Perspectives

Overall, the present example demonstrates that flavor composition F availability significantly increased water intake in healthy cats. Specifically, it was observed a 50% increase for the Regular diet and a 28% increase for the Urinary diet. Importantly, flavor composition F consumption varied according to individual cats, but was overall well accepted.

Flavor composition F consumption led overall to lower urine specific gravity, higher urine pH (+0.27 for Regular, and +0.17 for Urinary), identical RSS struvite, and lower RSS CaOx (−49% for Regular, and −33% for Urinary). Finally, it was observed that i) flavor composition F availability with Regular led to lower RSS CaOx, but higher RSS struvite, than when feeding a Urinary diet alone; and ii) flavor composition F availability with a Urinary diet improved the urinary performance (lower RSS CaOx and similar RSS struvite) compared to the Urinary diet alone.

In conclusion, the present example demonstrates that offering flavor composition F leads to similar RSS CaOx to offering a Urinary dry diet, but higher RSS struvite. Further, flavor composition F decreases RSS CaOx (and maintains RSS struvite) regardless of dry diet fed. Thus, the presently disclosed flavor compositions and pet food products disclosed herein can present and treat urinary tract disease.

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

1. A method of preventing and/or treating chronic kidney disease in an animal in need thereof, the method comprising administering a pet food product comprising
(a) a first amino acid capable of activating an umami receptor and a second amino acid not capable of activating the umami receptor; or
(b) a milk protein and a nucleotide or derivative thereof.

2. A method of preventing and/or treating urinary tract disease in an animal in need thereof, the method comprising administering a pet food product comprising
(a) a first amino acid capable of activating an umami receptor and a second amino acid not capable of activating the umami receptor; or
(b) a milk protein and a nucleotide or derivative thereof.

3. A method of increasing water consumption and/or hydration of an animal comprising administering a pet food product comprising a milk protein and a nucleotide or derivative thereof.

4-8. (canceled)

9. The method of claim 1, wherein
a) the first amino acid is alanine, histidine, glycine, serine, tyrosine, or a mixture thereof, and the second amino acid is proline, hydroxyproline, valine, threonine, lysine, or a mixture thereof;
b) the first amino acid is histidine and the second amino acid is proline, hydroxyproline, valine, threonine, lysine, or a mixture thereof; or
c) the first amino acid is alanine, histidine, glycine, serine, tyrosine, or a mixture thereof, and the second amino acid is proline.

10. The method of claim 9, wherein the first amino acid is histidine and the second amino acid is proline.

11. The method of claim 9, wherein the first amino acid and the second amino acid are at a molar ratio of about 1:1 or about 1:1.3.

12. (canceled)

13. The method of claim 9, wherein each of the first and second amino acids is in an amount of from about 1 mM to about 1 M, from about 20 mM to about 50 mM, or about 100 mM.

14-15. (canceled)

16. The method of claim 1, wherein
the pet food product does not comprise a phosphate or a derivative thereof; and/or
the pet food product does not comprise a furan or a derivate thereof.

17. The method of claim 1, wherein the pet food product further comprises a yeast extract.

18. The method of claim 3, wherein
(a) the milk protein is a casein or a derivative thereof; and/or
(b) the nucleotide or derivative thereof is AMP, UMP, GMP, IMP, CMP, or a mixture thereof.

19. The method of claim 3, wherein
(a) the milk protein is at an amount of from about 1% to about 10% by weight; and
(b) the nucleotide or derivative thereof is at an amount of from about 1 mM to about 100 mM.

20. The method of claim 19, wherein (a) the milk protein is at an amount of about 3% by weight, and/or (b) the nucleotide or derivative thereof is at an amount of about 5 mM.

21-24. (canceled)

25. The method of claim 1, wherein the nucleotide or derivative thereof is obtained from a yeast extract.

26. The method of claim 25, wherein the yeast extract is a Kluyveromyces yeast extract, a Torula yeast extract, or a Saccharomyces cerevisae yeast extract.

27-28. (canceled)

29. The method of claim 1, wherein the pet food product further comprises a furan or derivative thereof.

30. (canceled)

31. The method of claim 29, wherein the furan or derivative thereof is at an amount of from about 1 ppm to about 100 ppm.

32. (canceled)

33. The method of claim 1, wherein the pet food product further comprises a phosphate or derivative thereof, or a pyrophosphate.

34-39. (canceled)

40. The method of claim 1, wherein the pet food product is a packaged drinking water.

41. (canceled)

42. The method of claim 1, wherein the animal is a feline.

43-78. (canceled)

79. A flavor composition comprising:
a) a first amino acid capable of activating an umami receptor and a second amino acid not capable of activating the umami receptor;
b) a first amino acid capable of activating an umami receptor, a second amino acid not capable of activating the umami receptor, and a yeast extract;
c) a first amino acid capable of activating an umami receptor, a second amino acid not capable of activating the umami receptor, a yeast extract, a mineral, and a phosphate;
d) a first amino acid capable of activating an umami receptor, a second amino acid not capable of activating the umami receptor, a yeast extract, a mineral, a phosphate, and a thickening agent;
e) a milk protein and a nucleotide or derivative thereof;
f) a milk protein, a nucleotide or derivative thereof, and a furan or derivative thereof; or
g) a milk protein, a nucleotide or derivative thereof, a furan or derivative thereof, and a phosphate or derivative thereof.

80-88. (canceled)
